(19) United States
(12) Patent Application Publication
(10) Pub. No.: US 2003/0036184 A1
(43) Pub. Date: Feb. 20, 2003

(54) Polypeptides exhibiting PDE7 activity and their use for selecting compounds which inhibit PDE 7 enzyme activity

(76) Inventor: Patricia Soulard, Clamart (FR)

Correspondence Address: Mehdi Ganjeizadeh, Warner-Lambert Company, 2800 Plymouth Road, Ann Arbor, MI, 48105, US

(21) Appl. No.: 09/966,781

(22) Filed: Sep. 28, 2001

(30) Foreign Application Priority Data
Sep. 28, 2000 (EP) EP 0042026837

(51) Int. Cl. C12N 9/16; C07H 21/04; C12P 21/02; C12N 5/06

(52) U.S. Cl. 435/196; 435/69.1; 435/325; 435/320.1; 536/23.2

(57) ABSTRACT

The present invention relates to polypeptides exhibiting a higher PDE7 phosphodiesterase activity than the endogenous full length PDE7 and their use for selecting compounds which inhibit PDE7 enzyme activity.

### FIELD OF THE INVENTION

The present invention relates to novel polypeptides which exhibit phosphodiesterase 7 (PDE7) activity. These novel polypeptides are useful for the screening of PDE7 inhibitors.

### BACKGROUND OF THE INVENTION

Cyclic cAMP is an ubiquitous second messenger that plays a major role in signal transduction pathways and activates a family of cAMP-dependent protein kinases. The level of intracellular cAMP is regulated by a super family of enzymes known as phosphodiesterases (PDEs) whose main function is to degrade this second messenger to its inactivate metabolite, 5′ AMP. Therefore, the activity of these enzymes is critical in regulating the functional response of cells to a variety of extracellular agents such as hormones and neurotransmitters. PDEs constitute a complex group of enzymes divided into 11 families characterised by their structure, specificity against the substrates cAMP and cGMP, tissue expression, localisation and sensitivity to PDE inhibitors.

Four enzymes of these families, respectively PDE3, PDE4, PDE7 and PDE8 are highly selective for cAMP and are likely to be the most physiologically relevant cAMP metabolising enzymes.

The catalytic properties of PDE4 and PDE3 as well as their regulation have been extensively studied. However, little is known about the more recently discovered PDE7A. This enzyme is characterised by its specificity for cAMP, a high affinity for its substrate, with kinetic and pharmacological properties distinct from those of either PDE4 or PDE3.

Two splice variants exist for PDE7A, respectively PDE7A1 (Michaeli et al., 1993) and PDE7A2 (Bloom T. J. and Beavo J. A. et al., 1996), and the corresponding proteins differ only in their amino terminus sequence. PDE7A1 is a 55-57 kDa protein and is located predominantly in the soluble cellular fraction. PDE7A2 is a 53 kDa protein with a hydrophobic N-terminus and is found only in the particulate/membrane fraction of cells.

### SUMMARY OF THE INVENTION

The herein disclosed invention provides for the first time polypeptides derived from a full length PDE7A which surprisingly retain the catalytic activity of PDE7 and show a higher activity than the activity of the endogenous full length PDE7A1 and PDE7A2 proteins.

More particularly the herein disclosed invention provides for the first time polypeptides derived from a full length PDE7A which surprisingly show a higher activity than the activity of the endogenous full length PDE7A1 and PDE7A2 proteins.

The herein disclosed invention shows also for the first time the localisation of the catalytic site of the PDE7A enzyme.

The herein disclosed invention provides also for the first time the identification of the existence of a regulatory domain, more specifically of an inhibitory domain, in the sequence of PDE7, more specifically PDE7A, which appears to control or limit the catalytic activity of the full length PDE7 enzyme. Indeed, the inventors have shown that deletion of this regulatory domain significantly improved the catalytic activity of the resulting truncated PDE7A enzyme.

The recognition of the existence of this inhibitory/regulatory domain in PDE7(A) opens the possibility to improve the catalytic activity of PDE7(A) by blocking or reducing the impact of this inhibitory domain. In particular, the invention encompasses mutants of PDE7(A) which do not contain a portion of the PDE7(A) inhibitory domain which is essential to regulate the catalytic activity of the full length PDE7 enzyme. These mutants exhibit an improved PDE catalytic activity when compared to the full-length PDE7(A).

The invention concerns:

A polypeptide possessing a phosphodiesterase catalytic activity at least about 6 fold, preferably about 8 or 10 fold, most preferably 15 to 20 fold higher than the phosphodiesterase catalytic activity of an endogenous full length PDE7 protein which comprises at least the catalytic domain of the PDE7.

A polypeptide possessing a phosphodiesterase catalytic activity at least about 6 fold, preferably about 8 or 10 fold, most preferably 15 to 20 fold higher than the phosphodiesterase catalytic activity of an endogenous full length PDE7 protein which comprises at least the catalytic domain of the PDE7 with the exception of the amino acid sequence disclosed by Michaeli et al (1993) as the sequence consisting of the NH2 terminal deletion of HCP1 generated by sequential deletion of the sequence from the first ATG codon to residue 81 (L22M2).

The invention also regards a polypeptide as described above of up to about 427 amino acids in length possessing a phosphodiesterase 7 catalytic domain and comprising at least 312 consecutive amino acids of a sequence selected from the group consisting of the amino acid sequences of SEQ ID No 1, 2 or 3; or a homologous polypeptide thereof.

Said polypeptide comprises the aminoacid sequence which:

begins at the aminoacid residue located in position 1, 2, 3, 4, 5, 6, 7, 8, 9, 10, 11, 12,13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 of SEQ ID No1, 2 or 3; and which

ends at the aminoacid residue located in position 375, 376, 377, 378, 379, 380, 381, 382, 383, 384, 385, 386, 387, 388, 389, 390, 391, 392, 393, 934, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427 of SEQ ID No1, 2 or 3 or a homologous peptide thereof.

The polypeptides comprising the amino acid sequence beginning at the amino acid residue in position 5 and ending at the amino acid residue in position 427 of SEQ ID No1, 2 or 3; and/or those beginning at the amino acid residue in position 25 and ending at the amino acid residue in position 427 of SEQ ID No1, 2 or 3; and/or those beginning at the amino acid residue in position 45 and ending at the amino acid residue in position 394 of SEQ ID No 1, 2 or 3; and/or those beginning at the amino acid residue in position 45 and ending at the amino acid residue in position 427 of SEQ ID No1, 2 or 3 are preferred.

The polypeptides comprising the amino acid sequence beginning at the amino acid residue in position 45 and ending at the amino acid residue in position 394 of SEQ ID No 1, 2 or 3; and the polypeptide comprising the amino acid sequence beginning at the amino acid residue in position 45 and ending at the amino acid residue in position 427 of SEQ ID No1, 2 or 3 are most preferred.

The invention also concerns a polypeptide having at least 80% homology or identity, preferably 85% homology or identity with a polypeptide as defined above.

Most preferably the invention regards a polypeptide having at least 90% homology or identity, preferably 95% homology or identity, most preferably 99% homology or identity with a polypeptide as mentionned above.

Another subject of the invention is a nucleic acid sequence encoding a polypeptide as mentioned above, or a sequence complementary thereto.

A further aspect of this invention is a nucleic acid sequence which is selected from the group consisting of the nucleic acid sequences of SEQ ID No4, 5 or 6; or a sequence complementary thereto.

In addition to this, a nucleic acid sequence comprising a genomic polynucleotide encoding a PDE7 phosphodiesterase in which a portion of the nucleic acid encoding a polypeptide as described above has been deleted is also concerned in the present invention.

In an embodiment the invention concerns a nucleic acid sequence comprising the portion of the sequence of the full length PDE7 which remains after deletion of a nucleic acid sequence as described above.

In a preferred embodiment the full length PDE7 is of genomic origins.

The deleted nucleic acid portion of said nucleic acid is replaced by a heterologous polynucleotide sequence.

Said nucleic acid wherein the genomic polynucleotide encodes a mammalian PDE7 phosphodiesterase, preferably a human, mouse or rat PDE7 phosphodiesterase, most preferably a human PDE7 phosphodiesterase.

Said nucleic acid wherein the heterologous polynucleotide comprises a selection marker.

Said nucleic acid wherein the heterologous polynucleotide comprises at least a loxP sequence at its 5′ end and at least a loxp sequence at its 3′ end.

Said nucleic acid wherein the nucleic acid sequence encoding a polypeptide described above is operably linked to a regulatory sequence which is preferably an inducible promoter and is most preferably a promoter inducible by an inducer such as Ponasterone.

The invention also regards a recombinant vector comprising a nucleic acid sequence as described above, as well as a recombinant host cell comprising said nucleic acid.

The recombinant host cell may comprise a recombinant vector.

Said recombinant host cell may be eukaryotic and is selected from the group of recombinant hosts cell consisting of zygotes, embryonic, foetal or adult pluripotent stem cells, recombinant hosts cell derived from mammal blastomeres or blastocysts and ES cells.

Another aspect of the invention is a method for producing a polypeptide as described above, wherein said method comprises the steps of:

a) culturing, in an appropriate culture medium, a recombinant host cell described above;

b) harvesting the medium thus conditioned or lyse the recombinant host cell;

c) separating or purifying, from the said culture medium, or from the resultant cell lysate, the thus produced polypeptide.

The invention also embodies a method for the in vitro screening of a compound that inhibits PDE7 phosphodiesterase activity, wherein said method comprises the steps of:

a) providing a desired amount of a polypeptide as mentionned above;

b) adding the desired amount of a polypeptide provided in step a) to a buffer solution containing a desired amount of a candidate compound to be assayed;

c) measuring the phosphodiesterase enzyme activity; and

d) comparing the measured enzyme activity obtained at step c) with the enzyme activity obtained in the absence of the candidate compound.

Said method wherein the polypeptide provided at step a) consists of the amino acid sequence beginning at the amino acid residue in position 45 and ending at the amino acid residue in position 427 of SEQ ID No 1, or a homologous polypeptide thereof.

Said method wherein the polypeptide originates from the cell lysate of a host cell transfected with a nucleic acid as mentionned above or with a recombinant vector as described above.

Said method wherein the phosphodiesterase catalytic activity is measured as the level of hydrolysis of cAMP.

A method for the in vitro screening of a compound that inhibits PDE7 phosphodiesterase activity is also in the scope of the present invention, said method comprises the steps of:

a) Cultivating a recombinant host cell as described above in an appropriate culture medium;

b) adding a desired concentration of the candidate compound to be assayed in said culture medium;

c) measuring the intracellular phosphodiesterase enzyme activity; and

d) comparing the enzyme activity obtained at step c) with the enzyme activity obtained when step b) is omitted.

Said method wherein the recombinant host cell consists of a CHO cell line expressing the dimeric ecdysone receptor.

Said method wherein the recombinant host cell has been transfected with a recombinant vector comprising a nucleic acid encoding a polypeptide as described above which is operably linked to an inducible regulatory sequence, preferably the regulatory sequence is inducible by an inducer such as Ponasterone.

Said method wherein before step b), the recombinant hosts cell are cultivated in the presence of the inducer which activates the inducible regulatory sequence during step a).

Said method wherein an adenylate cyclase activator is added at the end of step b), before the addition of a stop solution.

Said method wherein the phosphodiesterase enzyme activity is measured after cell lysis through the measure of the amount of intracellular cAMP produced during step b).

The invention also embodies a method for the in vitro screening of a compound that inhibits PDE7 activity, wherein said method comprises the steps of:

a) providing a recombinant host cell coexpressing a polypeptide of the invention and a reporter gene, in an appropriate culture medium;

b) adding a desired concentration of the candidate compound to be assayed in said culture medium;

c) measuring the reporter gene expression; and

d) comparing the enzyme activity obtained at step c) with the enzyme activity obtained when step b) is omitted.

Said method wherein the recombinant host cell consists of a CHO cell line expressing the dimeric ecdysone receptor.

Said method wherein the recombinant host cell has been transfected with a recombinant vector comprising a nucleic acid encoding a polypeptide as described above which is operably linked to an inducible regulatory sequence.

Said method wherein the regulatory sequence is inducible by an inducer such as Ponasterone.

Said method wherein before step b), the recombinant hosts cell are cultivated in the presence of the inducer which activates the inducible regulatory sequence during step a).

Said method wherein an adenylate cyclase activator is added at the end of step b), before the addition of a stop solution.

Said method wherein the reporter gene is a beta lactamase gene.

Generally, the host cell cultivated at step a) may be any culturable host cell as mentioned above, and preferably a host cell of mammalian origin.

Another aspect of the present invention is a kit for the in vitro screening of a compound that inhibits PDE7 phosphodiesterase catalytic activity, wherein said kit comprises:

a) a polypeptide as described above;

b) optionally, the reagents necessary to perform the phosphodiesterase catalytic activity measures.

Another embodiment of the present invention is a kit for the in vitro screening of a compound that inhibits PDE7 phosphodiesterase catalytic activity, wherein said kit comprises:

a) a recombinant host cell as mentionned above;

b) optionally, the reagents necessary to perform the phosphodiesterase catalytic activity measurement.

A further embodiment of the present invention is a kit for the in vitro screening of a compound that inhibits PDE7 phosphodiesterase activity, wherein said kit comprises:

a) a recombinant host cell coexpressing a polypeptide as described above and a reporter gene; and

b) optionally, the reagents necessary to perform the reporter gene expression measurement.

Another aspect of the invention is a method or a process for selecting in vitro a compound that inhibits PDE7 phosphodiesterase activity, wherein said method comprises the steps of:

a) performing one of the method as described above with a candidate compound; and in case that said candidate compound is found to inhibit the phosphodiesterase activity, then

b) performing another method of the invention different of the method used with the inhibitor compound selected at step a)

A further aspect of the invention is a method for selecting a compound that selectively inhibits PDE7 phosphodiesterase activity, wherein said method comprises the steps of:

a) selecting a compound which inhibits PDE7 phosphodiesterase activity by carrying out a method as described above; and

b) assaying the selected inhibitor compound for its inability to inhibit the phosphodiesterase activity of at least one PDE enzyme other than PDE7.

Said method wherein the selected inhibitor is assayed for its inability to inhibit the phosphodiesterase activity of PDE3 and PDE4 enzymes, and preferably said method wherein the selected inhibitor is assayed for its inability to inhibit the phosphodiesterase activity of PDE1, PDE2, PDE3, PDE4, PDE5 and PDE6 enzymes.

The invention also regards a phosphodiesterase inhibitor compound selected according to a method as described above.

The invention also concerns the use of a phosphodiesterase inhibitor compound selected according to a method as mentionned above for manufacturing a pharmaceutical composition.

The present invention also encompasses a knock out animal generated by the use of a vector comprising a polynucleotide construct with a sequence comprising the portion of the full length PDE7 which remains after deletion of a nucleic acid as described above, said construct replacing thus a portion of the naturally occuring PDE7 sequence within the genome of this animal.

Preferably said knock out animal is of mammalian origin.

Most preferably said knock out animal is of murine or rat origin.

More preferably said knock out animal is of murine origin.

The invention also concerns:

1) A polypeptide possessing a phosphodiesterase catalytic activity at least about 6 fold, preferably about 8 or 10 fold, most preferably 15 to 20 fold higher than the phosphodiesterase catalytic activity of an endogenous full length PDE7 protein which comprises at least the catalytic domain of the PDE7 with the exception of the amino acid sequence disclosed by Michaeli et al (1993, J. Biol. Chem., 268, 12925-12932) as the sequence consisting of the NH2 terminal deletion of HCP1 generated by sequential deletion of the sequence from the first ATG codon to residue 81 (L22M2).

2) A polypeptide according to 1) above of up to about 427 amino acids in length possessing a phosphodiesterase 7 catalytic domain and comprising at least 312 consecutive amino acids of a sequence selected from the group consisting of the amino acid sequences of SEQ ID No 1, 2 or 3; or a homologous polypeptide thereof.

3) The polypeptide according to 2) above which comprises the aminoacid sequence which:

begins at the aminoacid residue located in position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 of SEQ ID No1, 2 or 3; and which

ends at the aminoacid residue located in position 375, 376, 377, 378, 379, 380, 381, 382, 383, 384, 385, 386, 387, 388, 389, 390, 391, 392, 393, 934, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427 of SEQ ID No1, 2 or 3 or a homologous peptide thereof.

4) The polypeptide according to 3) above which comprises the amino acid sequence beginning at the amino acid residue in position 5 and ending at the amino acid residue in position 427 of SEQ ID No1, 2 or 3; or a homologous polypeptide thereof.

5) The polypeptide according to 3) above which comprises the amino acid sequence beginning at the amino acid residue in position 25 and ending at the amino acid residue in position 427 of SEQ ID No1, 2 or 3; or a homologous polypeptide thereof.

6) The polypeptide according to 3) above which comprises the amino acid sequence beginning at the amino acid residue in position 45 and ending at the amino acid residue in position 427 of SEQ ID No1, 2 or 3; or a homologous polypeptide thereof.

7) The polypeptide according to 3) above which comprises the amino acid sequence beginning at the amino acid residue in position 5 and ending at the amino acid residue in position 394 of SEQ ID No1, 2 or 3; or a homologous polypeptide thereof.

8) The polypeptide according to 3) above which comprises the amino acid sequence beginning at the amino acid residue in position 25 and ending at the amino acid residue in position 394 of SEQ ID No1, 2 or 3; or a homologous polypeptide thereof.

9) The polypeptide according to 3) above which comprises the amino acid sequence beginning at the amino acid residue in position 45 and ending at the amino acid residue in position 394 of SEQ ID No 1, 2 or 3; or a homologous polypeptide thereof.

10) A polypeptide having at least 80% homology or identity, preferably 85% homology or identity, with a polypeptide as defined in anyone of 1) to 9) above.

11) A polypeptide having at least 90% homology or identity, preferably 95% homology or identity, most preferably 99% homology or identity with a polypeptide as defined in anyone of 1) to 9) above.

12) A polypeptide comprising the amino acid sequence which begins at an amino acid comprised between amino acids 57 to 100 and ends at an amino acid comprised between amino acids 450 to 483 of the full-length PDE7(A) protein with the exception of the amino acid sequence disclosed by Michaeli et al (1993, J. Biol. Chem., 268, 12925-12932) as the sequence consisting of the NH2 terminal deletion of HCP1 generated by sequential deletion of the sequence from the first ATG codon to residue 81 (L22M2).

13) A polypeptide according 12) above wherein the PDE7A protein is human PDE7A1 or PDE7A2.

14) A nucleic acid sequence encoding a polypeptide as defined in anyone of 1) to 13) above, or a sequence complementary thereto.

15) The nucleic acid sequence according to 14) above which is selected from the group consisting of the nucleic acid sequences of SEQ ID No4, 5 or 6; or a sequence complementary thereto.

16). A nucleic acid sequence comprising the portion of the sequence of the full length PDE7(A) which remains after deletion of a nucleic acid sequence according to 14) to 15) above.

17) A nucleic acid sequence according to anyone of 14) to 16) above which is of genomic origins.

18). A nucleic acid sequence according to 16) or 17) above wherein the deleted nucleic acid portion is replaced by a heterologous polynucleotide sequence.

19) A polypeptide according to anyone of 1) to 13) above or a nucleic acid d sequence according to anyone of 14) to 18) above, wherein PDE7(A) is of human, mouse or rat origin, most preferably human.

20) The nucleic acid according to 18) above, wherein the heterologous polynucleotide comprises a selection marker.

21) The nucleic acid according to 18) above, wherein the heterologous polynucleotide comprises at least a loxP sequence at its 5′ end and at least a loxP sequence at its 3′ end.

22) The nucleic acid of any one of anyone of 14) to 21) above, wherein the nucleic acid sequence encoding a polypeptide according to claim 2 is operably linked to a regulatory sequence.

23) A nucleic acid according to 22) above, wherein the regulatory sequence consists of an inducible promoter.

24) The nucleic acid sequence according to 23) above, wherein the regulatory sequence consists of a promoter inducible by an inducer such as Ponasterone.

25) A recombinant vector comprising a nucleic acid sequence as defined in 14) to 24) above.

26) A recombinant host cell comprising a nucleic acid as defined in 14) to 24) above.

27) A recombinant host cell comprising a recombinant vector according to 25) above.

28) A recombinant host cell according to 26) or 27) above which is eukaryotic.

29) The recombinant host cell according to anyone of 26) to 28) above which is selected from the group of recombinant hosts cell consisting of zygotes, embryonic, foetal or adult pluripotent stem cells, recombinant hosts cell derived from mammal blastomeres or blastocysts and ES cells.

30) A knock out animal generated by the use of a vector comprising a polynucleotide construct with a sequence comprising the portion of the full length PDE7 which remains after deletion of a nucleic acid according to anyone of 14) to 24) above, said construct replacing thus a portion of the naturally occuring PDE7 sequence within the genome of this animal.

31) A knock out animal according to 30) above of mammalian origin.

32) A knock out animal according to 31) above of murine or rat origin.

33) A method for producing a polypeptide as defined in 1) to 13) above, wherein said method comprises the steps of:

a) culturing, in an appropriate culture medium, a recombinant host cell as defined in 26) to 29) above;

b) harvesting the medium thus conditioned or lyse the recombinant host cell;

c) separating or purifying, from the said culture medium, or from the resultant cell lysate, the thus produced polypeptide.

34) A method for the in vitro screening of a compound that inhibits PDE7 phosphodiesterase activity, wherein said method comprises the steps of:

a) providing a desired amount of a polypeptide as defined in 1) to 13) above;

b) adding the desired amount of a polypeptide provided in step a) to a buffer solution containing a desired amount of a candidate compound to be assayed;

c) measuring the phosphodiesterase enzyme activity; and

d) comparing the measured enzyme activity obtained at step c) with the enzyme activity obtained in the absence of the candidate compound.

35) A method according to 34) above wherein the polypeptide provided at step a) consists of the amino acid sequence beginning at the amino acid residue in position 45 and ending at the amino acid residue in position 427 of SEQ ID NO 1, or a homologous polypeptide thereof.

36) A method according to 34) or 35) above wherein the polypeptide originates from the cell lysate of a host cell transfected with a nucleic acid as defined in 14) to 24) above or with a recombinant vector according to 25) above.

37) A method according to 34) or 36) above, wherein the phosphodiesterase catalytic activity is measured as the level of hydrolysis of cAMP.

38) A method for the in vitro screening of a compound that inhibits PDE7 phosphodiesterase activity, wherein said method comprises the steps of:

a) Cultivating a recombinant host cell as defined in 26) to 29) above in an appropriate culture medium;

b) adding a desired concentration of the candidate compound to be assayed in said culture medium;

c) measuring the intracellular phosphodiesterase enzyme activity; and

d) comparing the enzyme activity obtained at step c) with the enzyme activity obtained when step b) is omitted.

39) The method according to 38) above, wherein the recombinant host cell consists of a CHO cell line expressing the dimeric ecdysone receptor.

40) The method according to 38) above, wherein the recombinant host cell has been transfected with a recombinant vector comprising a nucleic acid encoding a polypeptide as defined in 1) to 13) above which is operably linked to an inducible regulatory sequence.

41) The method according to 40) above, wherein the regulatory sequence is inducible by an inducer.

42) The method according to 41) above, wherein the inducer is Ponasterone.

43) The method according to anyone of 38) to 42) above, wherein before step b), the recombinant hosts cell are cultivated in the presence of the inducer which activates the inducible regulatory sequence during step a).

44) The method according to 38) to 43) above, wherein an adenylate cyclase activator is added at the end of step b), before the addition of a stop solution.

45) The method according to 38) to 44) above, wherein the phosphodiesterase enzyme activity is measured after cell lysis through the measure of the amount of intracellular cAMP produced during step b).

46) A method for the in vitro screening of a compound that inhibits PDE7 activity, wherein said method comprises the steps of:

a) providing a recombinant host cell coexpressing a polypeptide according to anyone of 1) to 13) above and a reporter gene, in an appropriate culture medium;

b) adding a desired concentration of the candidate compound to be assayed in said culture medium;

c) measuring the reporter gene expression; and

d) comparing the enzyme activity obtained at step c) with the enzyme activity obtained when step b) is omitted.

47) The method according to 46) above, wherein the recombinant host cell consists of a CHO cell line expressing the dimeric ecdysone receptor.

48) The method according to 46) or 47) above, wherein the recombinant host cell has been transfected with a recombinant vector comprising a nucleic acid encoding a polypeptide as defined in 1) to 13) above which is operably linked to an inducible regulatory sequence.

49) The method according to 48) above, wherein the regulatory sequence is inducible by an inducer such as Ponasterone.

50) The method according to 46) to 49) above, wherein before step b), the recombinant hosts cell are cultivated in the presence of the inducer which activates the inducible regulatory sequence during step a).

51) The method according to 46) to 50) above, wherein an adenylate cyclase activator is added at the end of step b), before the addition of a stop solution.

52) the method of 46) to 51) above wherein the reporter gene is a beta lactamase gene.

53) A kit for the in vitro screening of a compound that inhibits PDE7 phosphodiesterase catalytic activity, wherein said kit comprises:

a) a polypeptide as defined in 1) to 13) above;

b) optionally, the reagents necessary to perform the phosphodiesterase catalytic activity measures.

54) A kit for the in vitro screening of a compound that inhibits PDE7 phosphodiesterase catalytic activity, wherein said kit comprises:

a) a recombinant host cell as defined in 26) to 29) above;

b) optionally, the reagents necessary to perform the phosphodiesterase catalytic activity measurement.

55) A kit for the in vitro screening of a compound that inhibits PDE7 phosphodiesterase activity, wherein said kit comprises:

a) a recombinant host cell of the invention coexpressing a polypeptide as defined in 1) to 13) above and a reporter gene; and

b) optionally, the reagents necessary to perform the reporter gene expression measurement.

56) A method for selecting in vitro a compound that inhibits PDE7 phosphodiesterase activity, wherein said method comprises the steps of:

a) performing the method as defined in 34) to 37) above with a candidate compound; and in case that said candidate compound is found to inhibit the phosphodiesterase activity, then

b) performing the method as defined in 38) to 52) above with the inhibitor compound selected at step a)

57) A method for selecting a compound that selectively inhibits PDE7 phosphodiesterase activity, wherein said method comprises the steps of:

a) selecting a compound which inhibits PDE7 phosphodiesterase activity by carrying out a method as defined in 34) to 37), 38) to 52) and 56) above; and

b) assaying the selected inhibitor compound for its inability to inhibit the phosphodiesterase activity of at least one PDE enzyme other than PDE7.

58) The method according to 57), wherein the selected inhibitor is assayed for its inability to inhibit the phosphodiesterase activity of PDE3 and PDE4 enzymes.

59) The method according to 57) above, wherein the selected inhibitor is assayed for its inability to inhibit the phosphodiesterase activity of PDE1, PDE2, PDE3, PDE4, PDE5 and PDE6 enzymes.

60) A phosphodiesterase inhibitor compound selected according to a method as defined in 34) to 37), 38) to 52), 56) and 57) to 59) above.

61) The use of a phosphodiesterase inhibitor compound selected according to a method as defined in 34) to 37), 38) to 52), 56) and 57) to 59) above. for manufacturing a pharmaceutical composition.

62) The use of a compound obtainable identifiable, selectable, or characterizable by the method according to anyone of 34) to 52) and 56) to

59) above for the manufacture of a pharmaceutical composition to be used in the treatment, diagnosis or surgery of the human or animal.

64) The use of a compound obtained, identified, selected or characterized by the method according to anyone of 34) to 52) and 56) to 59) above for the manufacture of a pharmaceutical composition to be used in the treatment, diagnosis or surgery of the human or animal.

65) The use of a compound obtained, identified, selected or characterized by the method according to anyone of 34) to 52) and 56) to 59) above for the manufacture of a pharmaceutical composition for the treatment or prevention of various pathological conditions such as diseases affecting the immune system, including AIDS, rejection of transplant, auto-immune disorders such as T-cells related diseases for example rheumatoid arthritis; inflammatory diseases such as respiratory inflammation diseases including chronic obstructive pulmonary disease (COPD), asthma; gastrointestinal inflammation diseases such as Crohn's disease, colitis, pancreatitis as well as different types of cancers including leukaemia.

66) The use of a compound obtainable, identifiable, selectable or characterizable by the method according to anyone of 34) to 52) and 56) to 59) above for the manufacture of a pharmaceutical composition for the treatment or prevention of various pathological conditions such as diseases affecting the immune system, including AIDS, rejection of transplant, auto-immune disorders such as T-cells related diseases for example rheumatoid arthritis; inflammatory diseases such as respiratory inflammation diseases including chronic obstructive pulmonary disease (COPD), asthma; gastrointestinal inflammation diseases such as Crohn's disease, colitis, pancreatitis as well as different types of cancers including leukaemia

67) The use of a compound obtained, identified, selected or characterized by the method according to anyone of 34) to 52) and 56) to 59) above in the synthesis of a compound for the treatment or prevention of various pathological conditions such as diseases affecting the immune system, including AIDS, rejection of transplant, auto-immune disorders such as T-cells related diseases for example rheumatoid arthritis; inflammatory diseases such as respiratory inflammation diseases including chronic obstructive pulmonary disease (COPD), asthma; gastrointestinal inflammation diseases such as Crohn's disease, colitis, pancreatitis as well as different types of cancers including leukaemia.

This invention does not concern the amino acid sequence disclosed by Michaeli et al (1993) as the sequence sequence consisting of the NH2 terminal deletion of HCP1 generated by sequential deletion of the sequence from the first ATG codon to residue 81 (L22M2).

### BRIEF DESCRIPTION OF THE FIGURES

FIG. 1 illustrates the alignment between the aminoacid sequences of two of the human PDE7 isoforms, respectively PDE7A1 (labelled h7A1.PRO) and PDE7A2 (labelled h7A2.PRO) as well as the mouse PDE7A2 (labelled mou7A2.PRO) and the rat PDE7A1 (labelled rat7.a.PRO) proteins. Aminoacid residues which aren't boxed denote aminoacid which are not homologous for the four aminoacids sequences.

FIG. 2 illustrates the structure of the different truncated PDE7 proteins obtained according to the invention. The upper line illustrates the human PDE7A1 full length isoform. The other lines illustrate the different truncated proteins tested. The numbers indicated in the left column denote the N-terminal and the C-terminal aminoacid residue of the protein, in reference to the full length PDE7A1 protein.

FIG. 3 illustrates the recombinant vector PcDNA4HisMax A which has been used for expressing the various PDE7 truncated proteins shown in FIG. 2.

As it can be seen in this figure, the DNA insert encoding the truncated PDE7 protein is inserted between the BamHI and the XbaI cloning site of said vector.

FIG. 4 illustrates results of PDE7A1 expression by recombinant CHO cell lines and Sf21 recombinant cell lines.

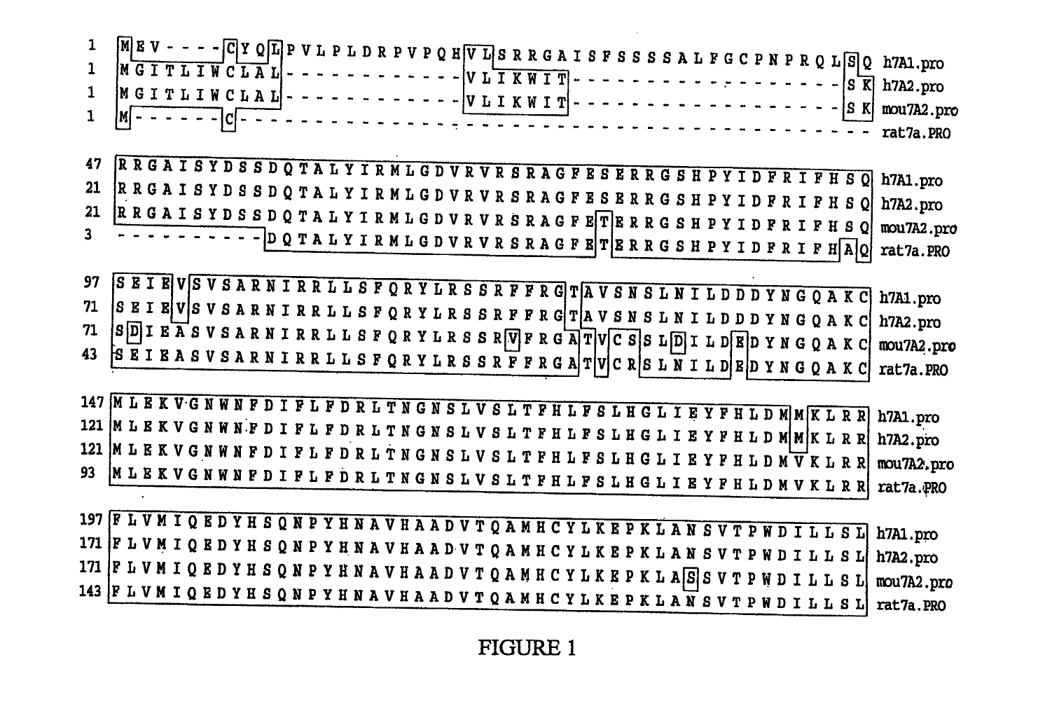

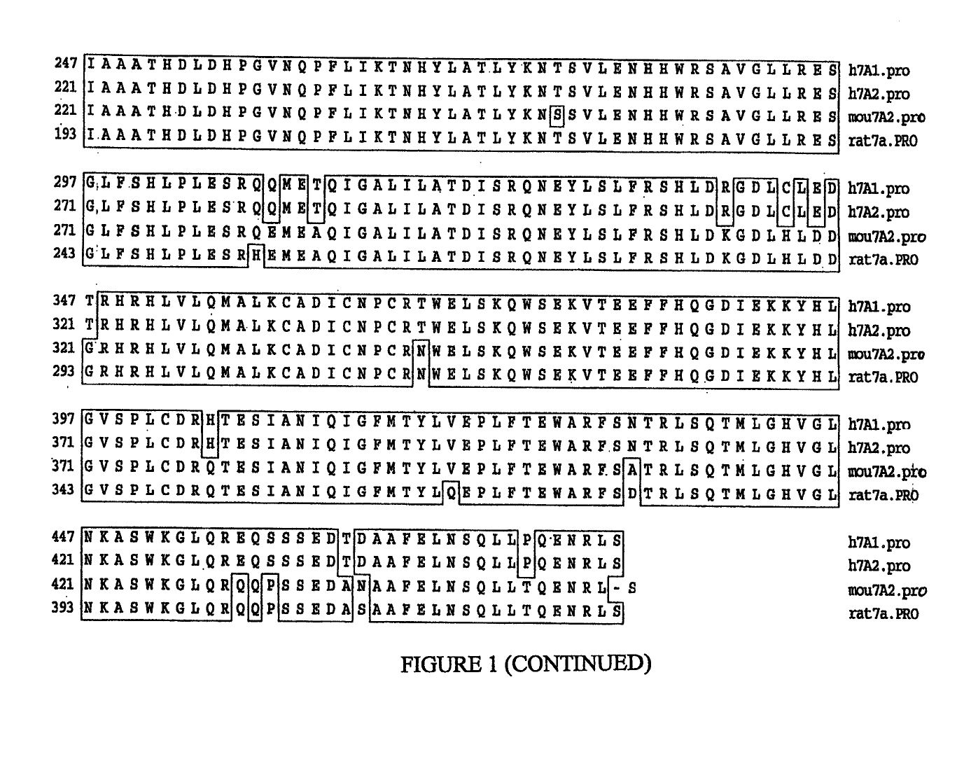

“Mocked CHO” denotes CHO cells which has been transfected with an empty recombinant vector.

“CHOrPDE7A1” denotes CHO cells transfected with a vector containing an insert coding for the human PDE7A1 protein.

“Sf21 rPDE7A1” denotes Sf21 cells transfected with a vector containing an insert coding for the human PDE7A1 protein.

“rPDE7A2” denotes recombinant CHO or Sf21 cells transfected with a vector containing an insert coding for the human PDE7A2 protein.

“rPDE4B2” denotes recombinant Sf21 cells transfected with a vector containing an insert coding for the human PDE4B2 protein.

The left bar of each group depicts the phosphodiesterase catalytic activity obtained without rolipram PDE 4 inhibitor.

The right bar of each group depicts the phosphodiesterase catalytic activity obtained in the presence of the rolipram PDE 4 inhibitor.

FIG. 5 illustrates the phosphodiesterase catalytic activity obtained respectively with CHO cells transfected with an empty vector, or with a vector containing an insert coding for PDE4B2 protein, PDE7A1 protein, PDE7A1 encoding insert comprising an optimal KOZAK sequence or PDE7A1 encoding insert comprising the cDNA 5′ and 3′ UTR.

FIG. 6 illustrates the phosphodiesterase catalytic activity expressed by recombinant CHO cells transfected respectively by an empty vector (controls), a vector encoding PDE4B2 protein, the full length PDE7A1 protein or the 13-483, 31-483, 61-483, 81-483, 101-483 and 171-483 truncated proteins (numbers referring to the full length PDE7A1 enzyme sequence).

FIG. 7 illustrates the phosphodiesterase catalytic activity expressed by recombinant CHO cells transfected respectively with an empty vector (controls), a vector encoding PDE4B2 protein, the PDE7A1 full length protein or the 101-483, 121-483, 141-483, 101-483, 101-450, 101-420 and 101-430 truncated PDE7A1 polypeptides. (numbers referring to the full length PDE7A1 enzyme sequence)

FIGS. 8A and 8B illustrate the construction of a targeting vector for homologous recombination which may be used to generate PDE7 knock-out mice.

FIGS. 9A and 9B represents a Western blot analysis of truncated forms of recombinant human PDE7A expression in CHO transfected cells.

FIG. 10 shows the dose response of beta lactamase expression in various EcrCHO clones expressing a preferred embodiment of a PDE7 catalytic site in presence of forskolin at various concentrations: the PDE7 mutant corresponding to the amino acids sequence 101-483 of the full length PDE7A. (“Fors” means forskolin).

FIG. 11 represents the measurement of beta lactamase expression in various EcrCHO clone 20 (30 000 cells) (n=2, ie the experiment has been made twice) expressing a preferred embodiment of a PDE7 catalytic site: PDE7 mutant corresponding to the amino acids sequence 101-483 of the full length human PDE7A1, after PDE inhibitors treatment.

FIG. 12 shows the PDE activity in CHO cells transfected with the nucleic acid encoding the PDE7 mutant corresponding to the amino acids sequence 101-483 of the full length human PDE7A1 (A1007A1) in presence or in absence of transfection of the nucleic acid encoding the first N-terminal 100 amino acids of the full length human PDE7A1 (Nter7A1).

FIG. 13 represents the PDE activity measurement in transfected CHO cells (lipofectamine) with a nucleic acid sequence encoding a preferred embodiment of a PDE7 catalytic site: the PDE7 mutant corresponding to the amino acids sequence 101-483 of the full length human PDE7A1 and a nucleic acid sequence encoding the first N-terminal 100 amino acids of the full length human PDE7A1 at different concentrations.

FIG. 14 represents a targeting vector for homologous recombination which may be used to generate a PDE7 knock-out C57BU6 mice.

FIG. 15 represents a targeting vector for homologous recombination which may be used to generate a PDE7 knock-out 129 mice.

### DETAILED DESCRIPTION OF THE INVENTION

The herein disclosed invention provides for the first time polypeptides derived from a full length PDE7 and preferably a full length PDE7A which surprisingly retain the catalytic activity of PDE7 and show a higher activity than the activity of the endogenous full length PDE7A1 and PDE7A2 proteins.

More particularly the herein disclosed invention provides for the first time polypeptides derived from a full length PDE7 and preferably a full length PDE7A which surprisingly show a higher activity than the activity of the endogenous full length PDE7A1 and PDE7A2 proteins.

The herein disclosed invention shows also for the first time the localisation of the catalytic site of the PDE7A enzyme.

The herein disclosed invention provides also for the first time the identification of the existence of a regulatory domain, more specifically of an inhibitory domain, in the sequence of PDE7, more specifically PDE7A, which appears to control or limit the catalytic activity of the full length PDE7 enzyme. Indeed, the inventors have shown that deletion of this regulatory domain significantly improved the catalytic activity of the resulting truncated PDE7A enzyme.

The recognition of the existence of this inhibitory/regulatory domain in PDE7(A) opens the possibility to improve the catalytic activity of PDE7(A) by blocking or reducing the impact of this inhibitory domain. In particular, the invention encompasses mutants of PDE7(A) which do not contain a portion of the PDE7(A) inhibitory domain which is essential to regulate the catalytic activity of the full length PDE7 enzyme. These mutants exhibit an improved PDE catalytic activity when compared to the full-length PDE7(A). Preferred mutants contain mutations in the N-terminal portion of PDE7(A) since the inhibitory/regulatory domain appears to be located at the N-terminus of the full-length PDE7(A). More specifically, the inhibitory/regulatory domain appears to be comprised between amino acids 31 and 100 of the full-length PDE7(A) protein. Hence, any mutation comprised between amino acids 31 and 100 of the full-length PDE7(A) protein results in an improvement of the activity of the corresponding PDE7(A) mutant with respect to the wild-type or full length PDE7(A). Thus, N-terminal deletion mutants of PDE7(A) beginning at an amino acid comprised between amino acids 57 to 100 and ending at an amino acid comprised between amino acids 450 to 483 of the full-length PDE7(A) protein have been generated. These mutants show a PDE7(A) activity or a PDE catalytic activity which is comprised between 6 and 20 fold the activity of the correponding full-length PDE7(A).

The primary aminoacid sequence of PDE7A is most closely related to the PDE4 family of PDEs, despite a relatively weak aminoacid identity. Indeed, the aminoacid sequence identity between PDE7A and PDE4 is of about 25% on the overall sequence. However, the aminoacid sequence identity between PDE7 and PDE4 reaches about 35% within the most conserved regions.

PDE7A is also characterized by its insensitivity to the standard selective inhibitors of other known PDE families including vinpocetin, SKF 94836, milrinone, rolipram or zaprinast. To date, no selective inhibitor of PDE7A has yet been reported.

Since the mRNA for PDE7A is widely found throughout various tissues and immune cells and because PDE7 enzyme activity has been detected in lymphocyte cells at a high level, it flows that PDE7A is potentially associated with several diseases. Those include diseases affecting the immune system, including AIDS, rejection of transplant, auto-immune disorders such as T-cells related diseases for example rheumatoid arthritis; inflammatory diseases such as respiratory inflammation diseases including chronic obstructive pulmonary disease (COPD), asthma; gastrointestinal inflammation diseases such as Crohn's disease, colitis, pancreatitis as well as different types of cancers including leukaemia.

There is thus a need for compounds which inhibit PDE7A enzyme activity. These would be of great pharmacological value as they could be useful for preventing or curing diseases associated with a modification in PDE7A cellular activity.

The selection of valuable inhibitor compounds of phosphodiesterase 7 activity requires the design of appropriate screening assays which are both highly sensitive and highly selective.

The inventors believe that highly sensitive screening assays for inhibitors of PDE7 would require polypeptides exhibiting a higher PDE7 catalytic activity than the catalytic activity observed for the naturally occurring PDE7A1 or PDE7A2 isoforms. Notably, polypeptides exhibiting a higher PDE7 catalytic activity than the naturally occurring PDE7A1 or PDE7A2 isoforms would be useful for selecting strong inhibitors of these enzymes.

The inventors also believe that the specificity of such a screening method for inhibitors of PDE7 enzyme could be reached if it could be ensured that a particularly selected compound interacts specifically with the catalytic domain of this enzyme.

According to the prior art, the catalytic domain of PDE7 is unknown and cannot be easily localised starting from the sole aminoacid sequence of PDE7. This is mainly due to the weak aminoacid homology of this enzyme with the other members of the PDE families, including PDE4. Furthermore according to Conti et al., 1995 sequencing of the different cDNAs encoding cAMP-PDEs has shown a puzzling heterogeneicity at the 5′ end of these clones.

The inventors have found that polypeptides derived from the full length PDE7A1 and PDE7A2 proteins and containing at least the catalytic domain characterised according to the invention, exhibit a Km value which is somewhat lower than the Km value observed for the naturally occurring PDE7A enzymes, and possess a great affinity for the cAMP substrate than the naturally occurring enzymes ie a higher Vmax substrate than the naturally occurring enzymes.

In addition, the inventors have now characterized novel polypeptides exhibiting PDE7 activity which mainly comprise the catalytic domain of this enzyme and which possess a Km value for the cAMP substrate which is of about 0.06 μM, whereas a Km value of about 0.2 μM and 0.12 μM observed for the PDE7A1 and PDE7A2 native proteins respectively (Ichimura M. and Kase H., 1993; Michaeli et al 1993; Bloom T. J. and Beavo J. A. et al., 1996).

Furthermore, it has been shown according to the present invention that the newly characterised polypeptides possess a phosphodiesterase catalytic activity which is at least 6, preferably 8 fold, more preferably 15 fold the catalytic activity observed for the endogenous full length PDE7A1 and PDE7A2 proteins and that some of the newly characterised polypeptides possess a phosphodiesterase catalytic activity which is about 15 to 20 fold the catalytic activity observed for the endogenous full length PDE7A1 and PDE7A2 proteins.

General Definitions of Biologically Relevant Terms

In accordance with the present invention there may be employed conventional molecular biology, microbiology, and recombinant DNA techniques within the skill of the art. Such techniques are explained fully in the literature. See, e.g., Sambrook et al., (1989); Glover, (1985); Gait, (1984); Hames and Higgins, (1985); Hames and Higgins, (1984); Freshney, (1986) and Perbal, (1984).

Therefore, if appearing herein, the following terms shall have the definitions set out below.

The term “isolated” for the purposes of the present invention designates a biological material (nucleic acid or protein) which has been removed from its original environment (the environment in which it is naturally present).

“Isolated polypeptide or protein” is substantially free of those compounds that are normally associated therewith in its natural state (e.g., other proteins or polypeptides, nucleic acids, carbohydrates, lipids).

“Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

For example, a polynucleotide present in the natural state in a plant or an animal is not isolated.

The term “purified” does not require the material to be present in a form exhibiting absolute purity, exclusive of the presence of other compounds. It is rather a relative definition.

A polynucleotide is in the “purified” state after purification of the starting material or of the natural material by at least one order of magnitude, preferably 2 or 3 and preferably 4 or 5 orders of magnitude.

For the purposes of the present description, the expression “nucleotide sequence” may be used to designate either a polynucleotide or a nucleic acid. The expression “nucleotide sequence” covers the genetic material itself and is therefore not restricted to the information relating to its sequence.

The terms “nucleic acid”, “polynucleotide”, “oligonucleotide” or “nucleotide sequence” cover RNA, DNA, gDNA, cDNA, synthetic DNA or semi-synthetic DNA sequences or alternatively RNA/DNA hybrid sequences of more than one nucleotide, either in the single-stranded form or in the duplex, double-stranded form.

The term “nucleotide” designates both the natural nucleotides (A, T, G, C) as well as the modified nucleotides that comprise at least one modification such as (1) an analog of a purine, (2) an analog of a pyrimidine, or (3) an analogous sugar, examples of such modified nucleotides being described, for example, in the PCT application No. WO 95/04 064.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome.

The term “homologous recombinant animal” as used herein is intended to describe an animal containing a gene which has been modified by homologous recombination between the gene and a DNA molecule introduced into an embryonic cell of the animal, or ancestor thereof. Thus, a homologous recombinant animal is a type of transgenic animal in which the transgene is introduced into a predetermined chromosomal location in the genome of the animal by homologous recombination.

“Regulatory region” or “regulatory sequence” means a nucleic acid sequence which regulates the expression of a nucleic acid. A regulatory region may include sequences which are naturally responsible for expressing a particular nucleic acid (a homologous region) or may include sequences of a different origin (responsible for expressing different proteins or even synthetic proteins). In particular, the sequences can be sequences of eukaryotic or viral genes or derived sequences which stimulate or repress transcription of a gene in a specific or non-specific manner and in an inducible or non-inducible manner. Regulatory regions include origins of replication, RNA splice sites, enhancers, transcriptional termination sequences, signal sequences which direct the polypeptide into the secretory pathways of the target cell, and promoters.

A regulatory region from a “heterologous source” is a regulatory region which is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art.

A “promoter sequence” is a DNA regulatory region capable of binding RNA polymerase in a cell and initiating transcription of a downstream (3′ direction) coding sequence. Within the promoter sequence is found a transcription initiation site (conveniently defined for example, by mapping with nuclease S1), as well as protein binding domains (consensus sequences) responsible for the binding of RNA polymerase.

A polypeptide is a polymeric compound comprised of covalently linked amino acid residues.

The “homologous” polypeptides of the invention include, but are not limited to, those containing, as a primary amino acid sequence, all or part of the amino acid sequence of a polypeptide according to the invention including altered sequences in which functionally equivalent amino acid residues are substituting residues within the sequence thus resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations are not expected to affect apparent molecular weight as determined by polyacrylamide gel electrophoresis, or isoelectric point.

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free CONH₂ can be maintained.

Amino acid substitutions may also be introduced to substitute an amino acid with a particularly preferable property. For example, a Cys may be introduced in a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

A “vector” is a replicon, such as plasmid, virus, phage, cosmid or bacmid, to which another DNA segment may be attached so as to bring about the replication of the attached segment. It is a circular or a linear DNA or RNA molecule which is either double-stranded or single-stranded. A “replicon” is any genetic element (e.g., plasmid, chromosome, virus) that functions as an autonomous unit of DNA replication in vivo, i.e., capable of replication under its own control.

Identity Between Two Nucleic Acid or Amino Acid Sequences

“Identity” refers to sequence identity between two peptides or between two nucleic acid molecules. Identity between sequences can be determined by comparing a position in each of the sequences which may be aligned for purposes of comparison. When a position in the compared sequences is occupied by the same base or amino acid, then the sequences are identical at that position. A degree of identity between nucleic acid sequences is a function of the number of identical nucleotides at positions shared by these sequences. A degree of identity between amino acid sequences is a function of the number of identical aminoacids at positions shared by these sequences. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for determining a comparison window may be conducted by the local homology algorithm of Smith and Waterman (1981), by the homology alignment algorithm of Needleman and Wunsch (1972), by the search for similarity method of Pearson and Lipman (1988), by computerized implementations of these algorithms (GAP, BESTFIT, FASTA, and TFASTA in the Wisconsin Genetics Solftware Package Release 7.0, Genetics Computer Group, 575, Science Dr. Madison, W1), or by inspection. The best alignment (i.e., resulting in the highest percentage of identity over the comparison window) generated by the various methods is selected. The term “sequence identity” means that two polynucleotide sequences are identical (i.e., on a nucleotide-by-nucleotide basis) over the window of comparison.

When not stated otherwise the positions of the amino acids in polypeptides are stated by reference to the human full length PDE7A1.

Polypeptides and Nucleic Acids of the Invention

Taking into account the high affinity for cAMP and the high phosphodiesterase catalytic activity of the newly characterized polypeptides of the invention as well as the fact that they comprise at least a catalytic domain of PDE7A enzymes, the polypeptides of the invention now allow the design of both highly sensitive and highly selective methods for the screening of PDE7A inhibitors.

As used herein the term catalytic domain or site means the minimal fragment required in order to obtain a PDE activity which is increased when compared with the PDE activity of the full length PDE7 enzyme.

Through aminoacid deletion experiments presented in the examples below, the inventors have shown that a polypeptide consisting of the sequence beginning at position 61 or 81 and ending at position 483 of the human PDE7A1 aminoacid sequence possesses a phosphodiesterase catalytic activity increased respectively by eight or nine fold as compared with the phosphodiesterase catalytic activity of the endogenous PDE7A1 or PDE7A2 full length proteins. Moreover, the inventors have also shown that a further increased phosphodiesterase catalytic activity could be observed for shorter polypeptides. For example, a polypeptide consisting of an aminoacid sequence starting at position 101 and ending at position 450 of the aminoacid sequence of human PDE7A1 exhibited a phosphodiesterase catalytic activity about 15 fold higher than the catalytic activity of the endogenous PDE7A1 and PDE7A2 full length proteins.

As used herein the term endogenous PDE7A1 or PDE7A2 means a PDE7A1 or PDE7A2 that is naturally expressed in mammalian cells.

Polypeptides

Consequently, a first object of the present invention is a polypeptide of up to about 427 aminoacids in length possessing a phosphodiesterase 7 catalytic domain and comprising at least 312 consecutive aminoacids of the sequence of SEQ ID No1, or a homologous polypeptide thereof.

An aminoacid sequence alignment between the human PDE7A1 and PDE7A2 aminoacid sequences and the PDE7 aminoacid sequences originating respectively from mouse and rat has shown a high aminoacid identity between the human and the mouse or the rat sequences. More precisely, between the aminoacid at position 57 and the aminoacid in position 483 of the human PDE7A1 protein, a 93,7% aminoacid identity with the corresponding mouse PDE7A2 aminoacid sequence and a 94,1% aminoacid identity with the rat PDE7A1 aminoacid sequence is observed.

Therefore, the aminoacid sequences derived respectively from the PDE7A mouse and rat aminoacid sequences and which share a high aminoacid identity with the human sequence of SEQ ID No1 also comprise the catalytic domain of the mouse or the rat PDE7 enzymes.

The mouse aminoacid sequence derived from the mouse PDE7A2 sequence and which aligns with the human sequence of SEQ ID No1 is referred to as the aminoacid sequence of SEQ ID No2.

The rat aminoacid sequence derived from the rat PDE7A1 sequence and which aligns with the human aminoacid sequence of SEQ ID No1 is referred to as the aminoacid sequence of SEQ ID No3.

A further object of the present invention consists of a polypeptide of up to about 427 aminoacids in length possessing a phosphodiesterase 7 catalytic domain and comprising at least 312 consecutive aminoacids of a sequence selected from the group consisting of the aminoacid sequences of SEQ ID No2 and 3, or a homologous polypeptide thereof.

For the purpose of the present invention, a “homologous polypeptide” encompasses polypeptides having at least 80%, more preferably at least 85%, most preferably 90 or 95 percent identity in aminoacids as regards the aminoacid sequences of SEQ ID No1, 2 and 3 above. The changes in aminoacid residues of a homologous polypeptide of the invention consist of aminoacid changes ranging from 1, 2, 3, 4, 5, 10 to 20 substitutions, additions or deletions of one aminoacid as regards the reference polypeptide.

In all cases, the “homologous polypeptide” exhibits a PDE7 phosphodiesterase catalytic activity which is at least of the same order of magnitude as the phosphodiesterase catalytic activity measured for the polypeptide having the aminoacid sequence of SEQ ID No1. In other words, a “homologous polypeptide” according to the invention possesses a phosphodiesterase activity of preferably at least six fold, and most preferably at least eight fold, the catalytic activity which is observed with the endogenous full length human PDE7A1 or PDE7A2 proteins.

The phosphodiesterase catalytic activity of anyone of the polypeptides encompassed by the invention may be assessed by one skilled in the art using the phosphodiesterase assays such as those described in the examples below.

A specific embodiment of a “homologous polypeptide” according to the invention includes a polypeptide molecule which is resistant to proteolysis, a peptide in which the —CONH— peptide bond is modified and replaced by (CH₂NH) reduced bond, a (NHCO) retro-inverso bond, a (CH₂—O) methylen-oxy bond, a (CH₂S) thiomethylene bond, a (CH₂CH₂) carba bond, a (CO—CH₂) cetomethylene bond, a (CHOH—CH₂) hydroxyethylene bond, a (N—N) bond and a —CH═CH— bond.

Another object of the invention is a polypeptide as defined above which comprises an aminoacid sequence which:

begins at the aminoacid residue located in position 1, 2, 3, 4, 5, 6, 7, 8, 9, 10, 11, 12, 13, 14, 15, 16, 17,18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 of SEQ ID No1, 2 or 3; and which

ends at the aminoacid residue located in position 375, 376, 377, 378, 379, 380, 381, 382, 383, 384, 385, 386, 387, 388, 389, 390, 391, 392, 393, 934, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427 of SEQ ID No1, 2 or 3 or a homologous peptide thereof.

Preferred polypeptides according to the invention are:

A polypeptide which comprises the aminoacid sequence beginning in position 5 and ending in position 427 of SEQ ID No1, 2 or 3, or a homologous polypeptide thereof.

A polypeptide which comprises the aminoacid sequence beginning in position 25 and ending in position 427 of SEQ ID No1, 2, or 3, or a homologous polypeptide thereof.

A polypeptide which comprises the aminoacid sequence beginning in position 45 and ending in position 427 of SEQ ID No1, 2 or 3 or a homologous polypeptide thereof.

A polypeptide which comprises the aminoacid sequence beginning in position 45 and ending in position 394 of SEQ ID No1, 2 or 3 or a homologous polypeptide thereof.

Most preferred polypeptides according to the invention are:

A polypeptide which comprises the aminoacid sequence beginning in position 45 and ending in position 427 of SEQ ID No1, 2 or 3 or a homologous polypeptide thereof.

A polypeptide which comprises the aminoacid sequence beginning in position 45 and ending in position 394 of SEQ ID No1, 2 or 3 or a homologous polypeptide thereof.

It has been shown according to the invention that the polypeptides 45-427 and 45-394 described above which are derived from the aminoacid sequence of SEQ ID No1, exhibit about 15 times more phosphodiesterase catalytic activity than the endogenous full length PDE7A1 and PDE7A2 proteins.

Nucleic Acids

A further object of the invention is a nucleic acid sequence which encodes a polypeptide of up to about 427 aminoacids in length possessing a phosphodiesterase 7 catalytic domain and comprising at least 312 consecutive aminoacids of a sequence selected from the group consisting of the aminoacid sequences of SEQ ID No1, 2 or 3; or a homologous polypeptide thereof.

The following preferred nucleic acids are encompassed by the present invention:

a nucleic acid encoding a polypeptide as defined above which comprises the aminoacid sequence which:

begins at the aminoacid residue located in position 1, 2, 3, 4, 5, 6, 7, 8, 9,10,11, 12,13, 14, 15, 16, 17,18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 of SEQ ID No1, 2 or 3; and which

ends at the aminoacid residue located in position 375, 376, 377, 378, 379, 380, 381, 382, 383, 384, 385, 386, 387, 388, 389, 390, 391, 392, 393, 934, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427 of SEQ ID No1, 2 or 3 or a homologous peptide thereof.

a nucleic acid encoding a polypeptide which begins at the aminoacid residue in position 5 and ends at the aminoacid residue in position 427 of SEQ ID No1, 2 or 3 or a homologous polypeptide thereof, and a nucleic acid sequence complementary thereto;

a nucleic acid encoding a polypeptide which begins at the aminoacid residue in position 25 and ends at the aminoacid residue in position 427 of SEQ ID No1, 2 or 3 or a homologous polypeptide thereof, and a nucleic acid sequence complementary thereto;

a nucleic acid encoding a polypeptide which begins at the aminoacid residue in position 45 and ends at the aminoacid residue in position 427 of SEQ ID No1, 2 or 3 or a homologous polypeptide thereof, and a nucleic acid sequence complementary thereto;

a nucleic acid encoding a polypeptide which begins at the aminoacid residue in position 45 and ends at the aminoacid residue in position 394 of SEQ ID No1, 2 or 3 or a homologous polypeptide thereof, and a nucleic acid sequence complementary thereto.

In a first preferred embodiment, the nucleic acid sequence is that of SEQ ID No4, which encodes the human polypeptide of SEQ ID No1, or a nucleic acid sequence complementary thereto.

In a second preferred embodiment, the nucleic acid sequence is that of SEQ ID No5, which encodes the mouse polypeptide of SEQ ID No2, or a nucleic acid sequence complementary thereto.

In a third preferred embodiment, the nucleic acid sequence is that of SEQ ID No6, which encodes the rat polypeptide of SEQ ID No3, or a nucleic acid sequence complementary thereto.

Preferably, anyone of the polypeptides and nucleic acid sequences according to the invention are under an isolated and/or purified form.

Vectors

Expression vectors

The nucleotide sequence coding for a polypeptide of the invention can be inserted into an appropriate expression vector, i.e., a vector which contains the necessary elements for the transcription and translation of the inserted protein-coding sequence. Such elements are termed herein a “promoter.” Thus, the nucleic acid encoding a polypeptide of the invention is operably linked to a promoter in an expression vector of the invention. Both cDNA and genomic sequences can be cloned and expressed under control of such regulatory sequences. An expression vector also preferably includes a replication origin.

The necessary transcriptional and translational signals can be provided on a recombinant expression vector.

In a first embodiment, a recombinant vector according to the invention is used to express a polypeptide as defined above which can be then purified.

In other embodiments, the expression vectors may be used for constructing transgenic animals. Preferred vectors are those containing a nucleic acid sequence comprising a genomic polynucleotide encoding a PDE7 phosphodiesterase which contain only a portion of the catalytic domain or none of it and wherein the deleted nucleic acid portions are replaced by a heterologous polynucleotide sequence.

Potential host-vector systems which can be used to express the sequences of the present invention include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant polypeptide of the invention, or a homologous polypeptide thereof, may be expressed chromosomally, after integration of the coding sequence by recombination. In this regard, any of a number of amplification systems may be used to achieve high levels of stable gene expression (See Sambrook et al., 1989).

Any of the methods described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

a) Promoters

Expression of a polypeptide of the invention may be controlled by any promoter/enhancer element known in the art, but these regulatory elements must be functional in the host selected for expression. Promoters which may be used to control the expression of the nucleic acid sequence encoding a polypeptide according to the invention include, but are not limited to, the SV40 early promoter region (Benoist and Chambon, 1981), the promoter contained in the 3′ long terminal repeat of Rous sarcoma virus (Yamamoto, et al., 1980), the herpes thymidine kinase promoter (Wagner et al., 1981), the regulatory sequences of the metallothionein gene (Brinster et al., 1982); prokaryotic expression vectors such as the β-lactamase promoter (Villa-Kaumaroff, et al., 1978), or the tac promoter (DeBoer, et al., 1983); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals.

A preferred promoter according to the invention is an inducible promoter. A most preferred promoter according to the invention is a promoter inducible by an inducer such as Ponasterone.

b) Host expression vector

A wide variety of host/expression vector combinations may be employed in expressing the nucleic acids of this invention. Suitable vectors include derivatives of known bacterial plasmids, e.g., Escherichia coli plasmids col El, pCR1, pBR322, pMal-C2, pET, PGEX (Smith et al., 1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2 m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Preferred host expression vector combination according to the invention are pGEX and PBR322.

Once a particular recombinant DNA molecule is identified and isolated, several methods known in the art may be used to propagate it. Once a suitable host system and growth conditions are established, recombinant expression vectors can be propagated and prepared in quantity.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992; Wu and Wu, 1988;Hartmut et al., Canadian Patent Application No. 2,012,311, filed Mar. 15, 1990).

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

c) Baculovirus Expression Systems

Expression systems can also include baculovirus in the context of the invention.

For example, in a baculovirus expression systems, both non-fusion transfer vectors, such as but not limited to pVL941 (BamHI cloning site; Summers), pVL1393 (BamHI, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamHI cloning site; Summers and Invitrogen), and pBlueBacIII (BamHI, BglII, PstI, NcoI, and HindIII cloning site, with blue/white recombinant screening possible; Invitrogen), and fusion transfer vectors, such as but not limited to pAc700 (BamHI and KpnI cloning site, in which the BamHI recognition site begins with the initiation codon; Summers), pAc701 and pAc702 (same as pAc700, with different reading frames), pAc360 (BamHI cloning site 36 base pairs downstream of a polyhedrin initiation codon; Invitrogen (195)), and pBlueBacHisA, B, C (three different reading frames, with BamHI, BglII, PstI, NcoI, and HindIII cloning site, an N-terminal peptide for ProBond purification, and blue/white recombinant screening of plaques; Invitrogen (220)) can be used.

Preferred transfer vectors in baculovirus expression systems according to the invention are BackPak (Clonetech) and pBlueBacHisA,B,C.

Another preferred type of recombinant transfer vectors to express the sequences of the invention consists of baculovirus vectors, such as the pVL 1392/1393 baculovirus transfer vector (Pharmingen) that may be used to transfect the SF9 cell line (ATCC NoCRL 1711) which is derived from Spodoptera frugiperda.

A most preferred baculovirus recombinant expression vector according to the invention consists of the AcMNPV vector (Invitrogen) which may be used to transfect the Sf21 cell line (Cawley P. et al, 1977).

Other suitable vectors for the expression of a polypeptide according to the invention in a baculovirus expression system include those described by CHAI et al. (1993), VLASAK (1983) and LENHARD et al. (1996).

d) Mammalian Expression Systems

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR (Kaufman et al., 1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech) can be used. In another embodiment, a vector that directs episomal expression under control of Epstein Barr Virus (EBV) can be used, such as pREP4 (BamHI, SfiI, XhoI, NotI, NheI, HindIII, NheI, PvuII, and KpnI cloning site, constitutive RSV-LTR promoter, hygromycin selectable marker; Invitrogen), pCEP4 (BamHI, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuII, NheI, HindIII, NotI, XhoI, SfiI, BamHI cloning site, inducible methallothionein IIa gene promoter, hygromycin selectable marker: Invitrogen), pREP8 (BamHI, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SmaI cloning site, TK- and b-gal selection), pMJ601 (SalI, SmaI, AflI, NarI, BspMII, BamHI, ApaI, NheI, SacII, KpnI, and HindIII cloning site; TK- and b-gal selection), and pTKgptF1S (EcoRI, PstI, SalI, AccI, HindII, SbaI, BamHI, and Hpa cloning site, TK or XPRT selection).

Preferred Mammalian expression vectors are pIND, pcDNA3, PcDNA4HisA,B,C,D.

e) Yeast Expression Systems

Yeast expression systems can also be used according to the invention to express an ABC1 polypeptide. For example, the non-fusion pYES2 vector (XbaI, SphI, ShoI, NotI, GstXI, EcoRI, BstXI, BamHI, SacI, Kpn1, and HindIII cloning sit; Invitrogen) or the fusion pYESHisA, B, C (XbaI, SphI, ShoI, NotI, BstXI, EcoRI, BamHI, SacI, KpnI, and HindIII cloning site, N-terminal peptide purified with ProBond resin and cleaved with enterokinase; Invitrogen), to mention just two, can be employed according to the invention.

Other preferred recombinant vectors according to the invention include expression vectors which are functional in mammalian cells which are well known by the one skilled in the art.

Homologous Recombination Vectors

Knock Out Animals

The determination by the inventors of the catalytic domain of the PDE7 enzymes now allows the design of polynucleotide constructs wherein the portion encoding the catalytic domain of a PDE7 enzyme, or a portion of it has been deleted.

In a preferred embodiment the polynucleotide construct as defined above contains a genomic polynucleotide encoding a PDE7 with non functional catalytic domain and wherein the deleted nucleic acid portion is replaced by a heterologous polynucleotide sequence.

Said constructs may be included in vectors in order to replace a portion of the naturally occurring PDE7 sequence within the genome of a mammal by homologous recombination.

According to this specific embodiment, such a recombinant vector of the invention may be used to generate transgenic knock-out animals, preferably transgenic knock-out mammals, most preferably transgenic knock-out mice and rats. Said transgenic knock-out mammals allowing a better model of drug action than a transgenic knock-out mammal generated via the deletion by homologous recombination of a nucleic acid portion bigger than the catalytic domain.

In order to isolate the genomic polynucleotides encoding human, mouse or rat PDE7 proteins, the one skilled in the art may refer respectively to:

the sequence of human PDE7A1 as disclosed in the GenBank/EBI database under the reference number L12052;

the sequence of human PDE7A2 as disclosed in the GenBank/EBI database under the reference number U67932;

the sequence of mouse PDE7A2 as disclosed in the GenBank/EBI database under the reference number U68171;

the sequence of mouse PDE7B as disclosed in the GenBank/EBI database under the reference number AJ251860 and

the sequence of rat PDE7A1 as disclosed in the GenBank/EBI database under the reference number U77880.

In a first embodiment of the nucleic acid above, the genomic polynucleotide encodes a human, a mouse or a rat PDE7 from which the catalytic domain or a portion of it has been deleted.

In a second embodiment of the nucleic acid above, the heterologous polynucleotide comprises a selection marker.

In a third embodiment of the nucleic acid above, the heterologous polynucleotide comprises at least a loxP sequence at its 5′ end and at least a loxP sequence at its 3′ end. The loxP sequence is composed of two palindromic sequences of 13 bp separated by a 8 bp conserved sequence (HOESS et al., 1986).

The recombination by the Cre enzyme between two loxP sites having an identical orientation leads to the deletion of the DNA fragment. The Cre-loxP system used in combination with a homologous recombination technique is described by GU et al. (1993, 1994).

The vector containing the genomic PDE7 sequence in which the sequence encoding the catalytic domain or a portion of it has been deleted is designed in such a way that selectable markers are flanked by loxP sites of the same orientation. It is possible, by treatment by the Cre enzyme, to eliminate the selectable markers while relocating the PDE7 genomic polynucleotide of interest that has been inserted by a homologous recombination event.

Two selectable markers are needed: a positive selection marker to select for the recombination event and a negative selection marker to select for the homologous recombination event. Vectors and methods using the Cre-loxP system are described by ZOU et al. (1994).

In the specific embodiment of the nucleic acids of the invention wherein said nucleic acid comprises the genomic polynucleotide encoding the mouse PDE7 phosphodiesterase in which the nucleic acid portion encoding its catalytic domain or a portion of it has been deleted, the person skilled in the art may advantageously refer to the examples below.

An illustrative construction of an “homologous recombination vector” according to the invention is shown in FIG. 8.

In a further aspect of the invention, a nucleic acid which encodes for a polypeptide as defined above is operably linked to a regulatory sequence.

Preferably, the regulatory sequence consists of a inducible promoter.

Most preferably, the regulatory sequence consists of a promoter inducible by an inducer such as Ponasterone.

Other Type of Homologous Recombinant Animal

Another type of homologous recombinant animal is an animal containing a gene encoding a PDE7 enzyme which has not been modified and in which a transgene encoding a polypeptide of the invention is added by homologous recombination. Thus in said homologous recombinant animal there is an overexpression of PDE7 enzyme.

Hosts Cells

Another object of the invention consists of a host cell that has been transformed or transfected with a nucleic acid according to the invention or which has been transformed or transfected with one of the recombinant vectors described above.

Thus, in a first embodiment, a recombinant host cell according to the invention comprises a nucleic acid encoding a polypeptide of up to about 427 aminoacids in length possessing a phosphodiesterase 7 catalytic domain and comprising at least 312 consecutive aminoacids of a sequence selected from the group consisting of the aminoacid sequences of SEQ ID No1, 2 or 3; or a homologous polypeptide thereof.

In a second embodiment, a recombinant host cell according to the invention comprises a nucleic acid sequence comprising a genomic polynucleotide encoding a PDE7 which does not contain a functional catalytic domain and wherein the deleted nucleic acid portion is replaced by a heterologous polynucleotide sequence.

Are included host cells that are transformed (prokaryotic cells) or that are transfected (eukaryotic cells) with a recombinant vectors such as one of those described herein.

Preferred host cells used as recipients for the expression vectors of the invention are the following:

a) prokaryotic host cells: Escherichia coli strains, Bacillus subtilis, or Salmonella typhimurium, Sf9 cells (ATCC NoCRL 1711), Sf21 cells (Cawley P. et al, 1977);

b) eukaryotic host cells: HeLa cells (ATCC NoCCL2), COS cells (ATCC NoCRL 1650; No CRL 1651) or CHO cells (ATCC NoCCL-61).

A most preferred cell line used for the expression of a polypeptide possessing a phosphodiesterase catalytic activity according to the invention consists of the CHO cell line expressing the dimeric ecdysone receptor (Invitrogen Inc.).

When the transfection of the host cell is carried out with a homologous recombination vector containing a nucleic acid sequence comprising a genomic polynucleotide encoding a PDE7 which does not contain a functional catalytic domain and wherein the deleted nucleic acid portion is replaced by a heterologous polynucleotide sequence, preferred hosts cell that may be used include mammalian zygotes, such as murine or rat zygotes such as disclosed by BRINSTER ET AL., 1985.

The vector can also be incorporated into embryonic foetal or adult pluripotent stem cells such as disclosed by CAPECCHI et al. (1991). Embryonic stem cells can be isolated from blastocysts or blastomeres cultivated in vitro and can be kept stable in culture over many cell generations, i.e. without any differentiation. Foreign DNA can be incorporated into the embryonic stem cells by electroporation. After selection of stem cells which carry the desired foreign DNA, the former are injected into the inner cell mass of blastocysts.

Preferred ES cell lines according to the invention are the following: ES-E14 TG2a (ATCC No CRL-1821) ES-D3 (ATCC NoCRL 1934 and NoCRL-11632), TS001 (ATCC NoCRL-11776), 36.5 (ATCC NoCRL-11116). To maintain ES cells in an uncommitted state, they are cultured in the presence of growth inhibited feeder cells which provide the appropriate signals to preserve this embryonic phenotype and serve as a matrix for ES cell adherence. Preferred feeder cells consist of primary embryonic fibroblasts that are established from tissue of day 13-day 14 embryos of virtually any mouse strain, that are maintained in culture, such as described by ABBONDANZO et al. (1993), or by the presence of an inhibitory concentration of LIF, such as described by PEASE and WILLIAMS (1990).

Methods/Processes for Selecting Compound Which Inhibit PDE7 Activity.

The novel polypeptides according to the invention, represent powerful tools to be included in a method for screening or selecting compounds which inhibit PDE7 enzyme activity.

Thus, a further object of the invention consists of a first method for the in vitro screening of a compound that inhibits PDE7 activity, wherein said method comprises the steps of:

a) providing a desired amount of a polypeptide of the invention, which comprises at least the catalytic domain of the PDE7 enzyme;

b) adding the desired amount of a polypeptide provided in step a) to a buffer solution containing a desired amount of a candidate compound to be assayed;

c) measuring the phosphodiesterase enzyme activity; and

d) comparing the measured activity obtained at step c) with the enzyme activity obtained in the absence of the candidate compound.

In a preferred embodiment of the first method of screening described above the polypeptide added at step a) consists of the amino acid sequence beginning at the amino acid residue in position 45 and ending at the amino acid residue in position 427 of SEQ ID No 1, or a homologous polypeptide thereof.

Preferably, the polypeptide of the invention which comprises at least the catalytic domain of the PDE7 protein is recombinantly produced by a recombinant host cell as defined above, most preferably by a recombinant CHO cell line or by a cell line derived from Spodoptera frugiperda like the Sf9 and the Sf21 cell lines.

Most preferably, the polypeptide of the invention which is used in the screening method above originates from the cell lysate of a recombinant host cell expressing said polypeptide. The cell lysate may be obtained for instance by sonication of the cultured recombinant host cell.

The polypeptide of the invention may be further purified, for example by high performance liquid chromatography, such as reverse phase and/or cationic exchange HPLC that are well known to those skilled in the art.

Alternatively, the polypeptide of the invention used for performing the screening method above may be prepared by conventional methods of chemical synthesis, either in a homogeneous solution or in solid phase.

As an illustrative embodiment of such chemical polypeptide synthesis techniques, it may be cited the homogeneous solution technique described by HOUBEN WEYL (1974). Said polypeptide may also be prepared according to the solid phase synthesis technique described by MERRIFIELD (1965a; 1965b).

According to the screening method above, the level of phosphodiesterase activity is determined through the measurement of the hydrolysis of the cAMP substrate of PDE7.

Preferably, the amount of a polypeptide of the invention which is added at step a) of the screening method above is defined as the amount leading to a final concentration in the assay mixture that causes less than 15% decrease in the initial concentration of substrate, such as for example less than a 15% decrease as regards the cAMP concentration before the addition of a polypeptide according to the invention. Preferably, the cAMP used as the enzyme substrate is radioactively labeled, for example under the form of [³H]-cAMP.

In a preferred embodiment of the screening method, the assay mixtures containing a desired amount of a polypeptide of the invention, either or not in the presence of a desired amount of a candidate inhibitor compound, and cAMP are incubated at 37° C. in a suitable buffer solution before the addition of a reagent that stops the enzyme reaction.

Preferably, the incubation time ranges from 5 to 20 min and is preferably of about 10 min.

Preferably, the reagent used to stop the enzyme reaction consists in silicate beads.

Preferably, the enzyme activity in each assay mixture is determined by measuring the level of radioactivity.

Then, phosphodiesterase catalytic activity measurements obtained in the presence of the candidate compound and those obtained when step b) is omitted (i.e. in the absence of the candidate compound) are compared and the IC50 value is determined. The IC50 value represents the concentration of the candidate compound tested which leads to a 50% inhibition of the cAMP hydrolysis, as compared with the cAMP hydrolysis obtained in control experiments performed in the absence of said candidate compound. The IC50 values may be calculated using a sigmoidal model with Hill slope well known by those skilled in the art.

The invention also pertains to a kit for the in vitro screening of a compound that inhibits PDE7 activity, wherein said kit comprises:

a) a polypeptide of the invention which comprises at least the catalytic domain of the PDE7 protein; and

b) optionally, the reagents necessary to perform the phosphodiesterase activity measurements.

Preferably, the polypeptide of the invention which is included in the kit above is in an aqueous solution or in the form of a lyophilised powder.

The reagents necessary for performing the phosphodiesterase catalytic activity measures include appropriate buffers and radio-labelled cAMP.

A further object of the present invention consists of a second method for the in vitro screening of a compound that inhibits PDE7 activity, wherein said method comprises the steps of:

a) providing a recombinant host cell expressing a polypeptide of the invention which comprises at least the catalytic domain of PDE7 in an appropriate culture medium;

b) adding a desired concentration of the candidate compound to be assayed in said culture medium;

c) measuring the intracellular phosphodiesterase enzyme activity and

d) comparing the enzyme activity obtained at step c) with the enzyme activity obtained when step b) is omitted.

Generally, the host cell cultivated at step a) may be any culturable host cell as mentioned above, and preferably a host cell of mammalian origin.

In a first preferred embodiment of the screening method above, the recombinant host cell consists of a CHO cell line expressing the dimeric ecdysone receptor.

In another preferred embodiment of the screening method above, the recombinant host cell used has been transfected with a recombinant vector comprising a nucleic acid encoding a polypeptide according to the invention wherein said nucleic acid is operably linked to an inducible regulatory sequence.

In this preferred embodiment, the inducer compound towards which the inducible regulatory sequence is responsive may be added at step a) of the screening method in order to allow a high expression level of the polypeptide of the invention, thus increasing the sensitivity of the screening method.

Most preferably, the inducible regulatory sequence is responsive to Ponasterone such as the one contained in the inducible vector pIND (In Vitrogen).

According to the screening method above, the recombinant hosts cell are cultivated in the presence of the inducer compound which activates the inducible regulatory sequence during step a).

Advantageously, the incubation time of the hosts cell in the presence of the inducer compound is comprised between 1 and 20 hours, preferably between 4 and 15 hours and most preferably between 6 and 10 hours.

After induction, the culture medium is removed and fresh medium is added for carrying out the assay. The candidate compounds are added to the fresh medium at desired concentrations (e.g. series of increasing concentrations) and the cells are incubated at 37° C. for a period of time ranging from 10 to 60 minutes, preferably 20 to 45 minutes and most preferably during about 30 minutes.

Preferably, the reaction is ended by the addition of an adenylate cyclase activator at the end of step b), before the addition of a stop solution.

Most preferably, the adenylate cyclase activator consists of the compound forskolin.

The incubation time with the adenylate cyclase activator advantageously ranges between 5 and 30 minutes, preferably between 10 and 20 minutes and most preferably during about 15 minutes.

According to the second screening method described above, the phosphodiesterase enzyme activity is measured after cell lysis through the determination of the amount of intracellular cAMP produced during step b).

The measurement of the cAMP levels in the cell lysate may be carried out using an antibody directed to cAMP.

Most preferably, the measurement of the amount of intracellular cAMP produced during step b) is performed in the cell lysate using a polyclonal antibody directed to cAMP in an competition assay wherein cAMP in the cell lysate competes with an alkaline phosphatase molecule which has cAMP covalently attached thereto for binding to said anti-cAMP polyclonal antibody.

Most preferably, the cellular cytosol is separated from the particulate fraction of the cell lysate, for example by centrifugation, before measuring the cAMP content on the cytosolic fraction such as described above.

Then, the values of intra-cellular cAMP measured in the presence of increasing concentrations of the candidate compound assayed and the cAMP measures when step b) is omitted (i.e. in the absence of the candidate compound) are compared and the AUC (area under the curve) value for the candidate compound is determined.

The AUC value for a given candidate compound represents the concentration of said candidate compound leading to an increase of 100 to 2000% of the amount of intracellular cAMP produced during step b) of the screening method. The invention also pertains to a kit for the in vitro screening of a compound that inhibits PDE7 phosphodiesterase activity, wherein said kit comprises:

a) a recombinant host cell of the invention expressing a polypeptide of the invention; and

b) optionally, the reagents necessary to perform the phosphodiesterase catalytic activity measurement. In another embodiment of the above second method for the in vitro screening of a compound that inhibits PDE7 activity, said method comprises the steps of:

a) providing a recombinant host cell coexpressing a polypeptide of the invention which comprises at least the catalytic domain of PDE7 in an appropriate culture medium and a reporter gene;

b) adding a desired concentration of the candidate compound to be assayed in said culture medium;

c) measuring the reporter gene expression; and

d) comparing the enzyme activity obtained at step c) with the enzyme activity obtained when step b) is omitted.

Generally, the host cell cultivated at step a) may be any culturable host cell as mentioned above, and preferably a host cell of mammalian origin.

In a first preferred embodiment of the screening method above, the recombinant host cell consists of a CHO cell line expressing the dimeric ecdysone receptor.

In another preferred embodiment of the screening method above, the recombinant host cell used has been transfected with a recombinant vector comprising a nucleic acid encoding a polypeptide according to the invention wherein said nucleic acid is operably linked to an inducible regulatory sequence.

In this preferred embodiment, the inducer compound towards which the inducible regulatory sequence is responsive may be added at step a) of the screening method in order to allow a high expression level of the polypeptide of the invention, thus increasing the sensitivity of the screening method.

Most preferably, the inducible regulatory sequence is responsive to Ponasterone such as the one contained in the inducible vector pIND (In Vitrogen).

Most preferably the reporter gene is the beta lactamase gene.

According to the screening method above, the recombinant hosts cell are cultivated in the presence of the inducer compound which activates the inducible regulatory sequence during step a).

Advantageously, the incubation time of the hosts cell in the presence of the inducer compound is comprised between 1 and 20 hours, preferably between 4 and 15 hours and most preferably between 6 and 10 hours.

After induction, the culture medium is removed and fresh medium is added for carrying out the assay. The candidate compounds are added to the fresh medium at desired concentrations (e.g. series of increasing concentrations) and the cells are incubated at 37° C. for a period of time ranging from 10 to 60 minutes, preferably 20 to 45 minutes and most preferably during about 30 minutes.

Preferably the cell concentration used is between 15000 and 40000 cells per well, most preferably between 30000 and 40000 cells per well and more preferably 30000 cells per well.

Preferably, the reaction is ended by the addition of an adenylate cyclase activator at the end of step b), before the addition of a stop solution.

Most preferably, the adenylate cyclase activator consists of the compound forskolin.

The incubation time with the adenylate cyclase activator advantageously ranges between 5 and 30 minutes, preferably between 10 and 20 minutes and most preferably during about 15 minutes.

The invention also pertains to a kit for the in vitro screening of a compound that inhibits PDE7 phosphodiesterase activity, wherein said kit comprises:

a) a recombinant host cell of the invention coexpressing a polypeptide of the invention and a reporter gene; and

b) optionally, the reagents necessary to perform the reporter gene expression measurement.

The first in vitro screening method described above makes use of a polypeptide of the invention which comprises at least the PDE7 catalytic domain contained in a buffer solution. In a specific embodiment of the invention, said first in vitro screening method is used as a primary screening test for inhibitors of PDE7 or phosphodiesterase activity.

A candidate compound which has been positively screened according to said first screening method of the invention may then be tested according to the second screening method described above which makes use of recombinant hosts cell expressing a polypeptide of the invention which comprises at least the PDE7 catalytic domain. Said second screening method allows the selection of PDE7 inhibitors in a cellular, thus in a physiological environment.

Therefore, the invention also deals with a method for selecting in vitro a compound that inhibits PDE7 activity, wherein said method comprises the steps of:

a) performing the first screening method of the invention described above with a candidate compound; and if said candidate compound is found to inhibit phosphodiesterase activity, then

b) performing the second screening method according to the invention with the inhibitor compound selected at step a).

There is no need to say that candidate compounds which are positively selected according to the method above which combines the first and second screening methods of the invention represent potential drugs of therapeutic value.

According to the invention, it may also be ensured that the PDE7 inhibitor compounds selected according to any one of the screening or selection methods above is selective for PDE7 and does not inhibit one or several other phosphodiesterase enzymes.

Thus, the invention also pertains to a method for selecting a compound that selectively inhibits PDE7 activity, wherein said method comprises the steps of:

a) selecting a compound which inhibit PDE7 phosphodiesterase activity by carrying out the first and/or the second screening method defined above; and

b) assaying the selected inhibitor compound for its inability to inhibit the phosphodiesterase activity of at least one PDE enzyme other than PDE7.

In a first preferred embodiment of the selection method above, the selected inhibitor is assayed for its inability to inhibit the phosphodiesterase activity of the PDE3 and PDE4 enzymes.

In a second preferred embodiment of the selection method above, the selected inhibitor is assayed for its inability to inhibit the phosphodiesterase activity of the PDE1, PDE2, PDE3, PDE4, PDE5 and PDE6 enzymes.

As an illustrative example, the person skilled in the art may advantageously refer to the article of TORPHY et al. (1993) or to the article of KOVALA et al. (1997) which both disclose methods for determining the catalytic activity of PDE4.

The person skilled in the art may advantageously refer to the article of Manganellio et al. that discloses a method for measuring the catalytic activity of PDE3.

The one skilled in the art may advantageously refer to the article of FINK et al. (1999), which discloses an assay for measuring the catalytic activity of PDE5.

The one skilled in the art may advantageously refer to the article of SONNENBURG et al. (1995), which discloses an assay for determining the catalytic activity of PDE1.

The one skilled in the art may advantageously refer to the article of BEAVO et al. (1970), which discloses an assay for determining the catalytic activity of PDE2.

The one skilled in the art may advantageously refer to the articles of BAEHR et al. (1979) and FUNG et al. (1990), which disclose an assay for determining the catalytic activity of PDE6.

The invention also concerns a PDE7 inhibitor compound which has been selected according to any one of the methods described above.

The invention further relates to the use of a PDE7 inhibitor compound which has been selected according to any one of the methods described above for manufacturing a pharmaceutical composition.

This invention does not concern the amino acid sequence disclosed by Michaeli et al (1993) as the sequence sequence consisting of the NH2 terminal deletion of HCP1 generated by sequential deletion of the sequence from the first ATG codon to residue 81 (L22M2).

The present invention is further illustrated, but in no case limited, by the figures and the examples below.

### EXAMPLES

A. Materials and Methods

Material

Restriction enzymes were purchased from Roche Molecular Biochemicals. Protein concentrations were determined according to the method of Bradford using BSA as standard. Sequencing was performed on an automated sequencing machine (ABI model 373; Perkin Elmer) using 2 μg of plasmid DNA and 5 pmol of appropriate sequencing oligonucleotide (DNA sequencing Kit, Dye terminator Cycle Sequencing Ready Reaction, Perkin Elmer). SDS/PAGE was performed according to the method of Laemmli (1970).

Cloning of PDE7A 1 cDNA

Total RNA was extracted from human lymphocyte T cells (CD4+) using reagents from Stratagene cloning systems kit (La Jolla Calif., USA) according to the instructions of the manufacturer. cDNA was synthesized from 10 μg of total RNA using oligo d(T) primer and MMLV reverse transcriptase in a final volume of 50 μl (Roche-Molecular System Branchburg, N.J.). 1 μl of the first strand cDNA product was then used as template for PCR amplification using Amplitaq cDNA polymerase (Roche-Molecular System, Branchburg, N.J.) to generate the cDNA encoding human PDE7A1 using oligonucleotides designed from the published sequence (Michaeli et al., 1997; Bloom and Beavo, 1996) which flank the open reading frame. The primers had the following sequences:

| | | |
|---|---|---|
| PDE7A1 | CGGGGATCCATGGAAGTGTGTTACCAG | (SEQ ID N^{o} 12) |
| PDE7A2 | CGCGTCTAGATTATGATAACCGATTTTCCTGAGGTAA | (SEQ ID N^{o} 13) |

The restriction enzyme sites BamHI and XbaI underlined in the above primers were used to subclone the PCR product into the baculovirus expression vector, pBlueBacHisA, or pcDNA4 vector (In vitrogen, San Diego, Calif.). The PCR amplification product of PDE7A coding sequence was placed adjacent to the tag, 6xHis, to create a fusion protein with the tag at the amino terminus of the recombinant enzyme. All constructs were sequenced and checked prior to use. These clones were named pcDNA4his7A1 and pBacHis7A1.

The complete open reading frame of PDE7A1 was also cloned into pcDNA3 containing the Kozak sequence, GCCACCATG (Kozak M., JBC, 266,1991) which was added immediately upstream of the initiation codon of PDE7A1. This clone was named pcDNAKoz7A. The primers had the following sequences:

| | | |
|---|---|---|
| PDE7AK1 | CGGGATCCGCCACCATGGAAGTGTGTTACC | (SEQ ID N^{o} 14) |
| PDE7AK2 | CGCGTCTAGATTATGATAACCGATTTTCCTGAGGTAA | (SEQ ID N^{o} 13) |

In addition, the 3′ and 5′ untranslated region (UTR) sequences of PDE7A1 (Michaeli et al., (1997) were added to the full open reading frame of PDE7A1 in pcDNA3. Thus, 1050 bp of PDE7A1 3′ UTR and 152 bp of PDE7A2 5′UTR were amplified by RT-PCR, from cDNA derived from human CD4+ T cells using the following primers:

| | | |
|---|---|---|
| UCR3′1 | CCGGGGGTACCGGCGGCCGCGGCAGGGCGGGCGCCG | (SEQ ID N^{o} 15) |
| UCR3′2 | GGGGGATCCTGAATACGCCCGCCCTGCCTCCG | (SEQ ID N^{o} 16) |
| UCR5′1 | GGGTCTAGACCCCAGAACCAGTGGGACAAACTGCCTCCT | (SEQ ID N^{o} 17) |
| UCR5′2 | GGCGGGCCCTGGAGAAACATAACATGCACGTCAC | SEQ ID N^{o} 18) |

The 5′ UTR sequence was added immediately upstream of the initiation codon and the 3′ UTR sequence downstream of the stop codon of the PDE7A1 coding sequence. The restriction enzyme sites underlined in the above primers are respectively KpnI, BamHI, XbaI and SmaI and were used to facilitate subcloning into pcDNAKoz7A. This clone was named pcDNA3′5′7A.

The sequence of all constructs were verified prior to use.

Construction of Amino and Carboxy Terminal Deletions of PDE7A

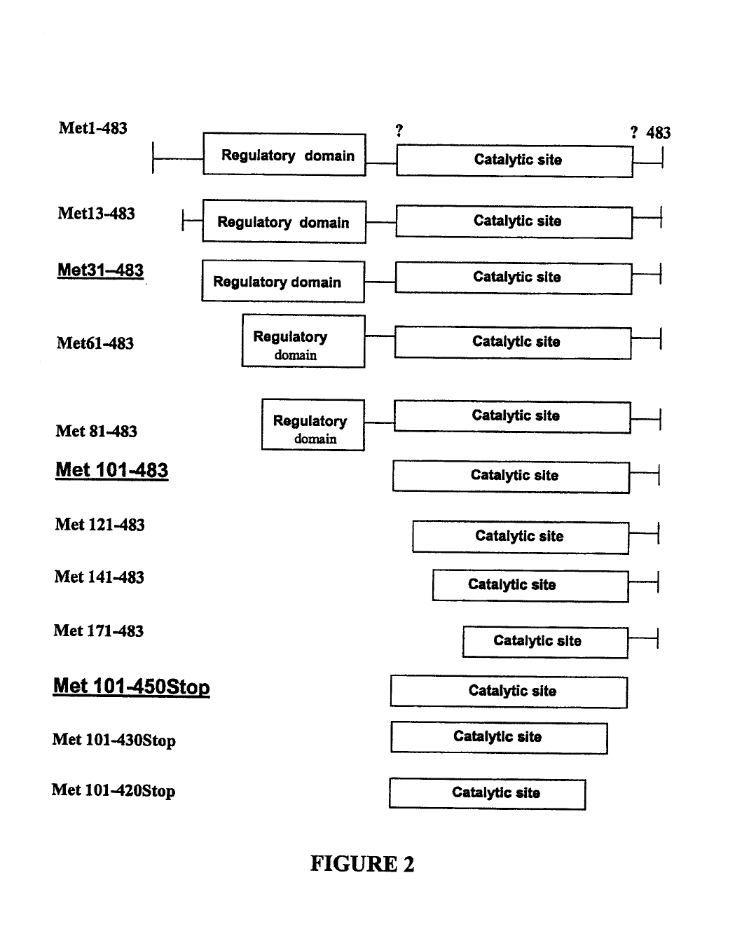

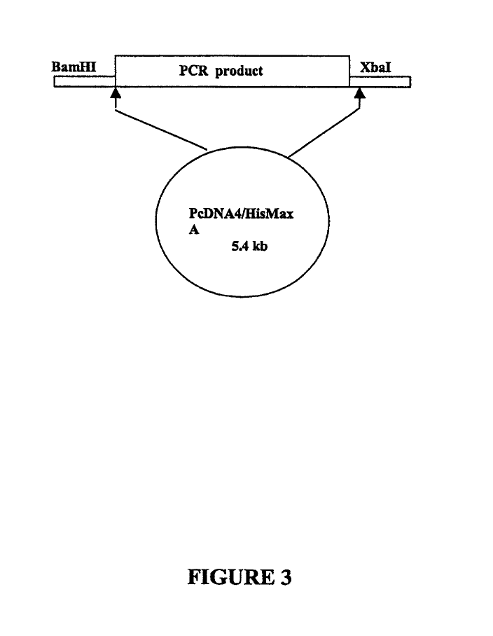

The N and C terminal regions of PDE7A1 were progressively deleted to map the catalytic domain and to identify a putative inhibitory domain. Eleven constructs (FIG. 2) were generated by PCR amplification using the full length PDE7A1 cDNA as template and primers which contained restriction sites (BamHI and XbaI) to allow subcloning into BamHI/XbaI site of pcDNA4 (Invitrogen) downstream from histidine tag.

Amino Terminal Deletions

The following primers were used to make the construct PDE7A12 which lacks the first 12 amino acids to generate PDE7A;13-483.

| | |
|---|---|
| H1 | |
| CCGGGATCCGACAGGCCGGTCCCCCAGCACGTCCTC | (SEQ ID N^{o} 19) |
| H2 | |
| CGCGTCTAGATTATGATAACCGATTTTCCTGAGG | (SEQ ID N^{o} 20) |

The following primers were used to make the construct PDE7Δ30 which lacks the first 30 amino acids to generate PDE7A;31-483.

| | |
|---|---|
| A1 | |
| CCGGGATCCCCCCGGCAGCTCTCTCAGAGGCGT | (SEQ ID N^{o} 21) |
| H2 | |
| CGCGTCTAGATTATGATAACCGATTTTCCTGAGG | (SEQ ID N^{o} 20) |

The following primers were used to make the construct PDE7Δ60 which lacks the first 60 amino acids to generate PDE7A;61-483.

| | |
|---|---|
| B1 | |
| CCGGGATCCTTATACATTCGTATGCTAGGAG | (SEQ ID N^{o} 22) |
| H2 | |
| CGCGTCTAGATTATGATAACCGATTTTCCTGAGG | (SEQ ID N^{o} 20) |

The following primers were used to make the construct PDE7Δ80 which lacks the first 80 amino acids to generate PDE7A;81-483.

| | |
|---|---|
| C1 | |
| CCGGGATCCAGAAGAGGTTCTCACCCATATA | (SEQ ID N^{o} 23) |
| H2 | |
| CGCGTCTAGATTATGATAACCGATTTTCCTGAGG | (SEQ ID N^{o} 20) |

The following primers were used to make the construct PDE7Δ100 which lacks the first 100 amino acids to generate PDE7A;101-483.

| | |
|---|---|
| D1 | |
| CCGGGGATCCGTGTCTGTCTCTGCAAGGAATATC | (SEQ ID N^{o} 24) |
| AGA | |
| H2 | |
| CGCGTCTAGATTATGATAACCGATTTTCCTGAGG | (SEQ ID N^{o} 20) |

The following primers were used to make the construct PDE7Δ120 which lacks the first 120 amino acids to generate PDE7A;121-483.

| | |
|---|---|
| E1 | |
| CCGGGGATCCATGTCACGCTTTTTTCGTGGTACTG | (SEQ ID N^{o} 25) |
| H2 | |
| CGCGTCTAGATTATGATAACCGATTTTCCTGAGG | (SEQ ID N^{o} 20) |

The following primers were used to make the construct PDE7Δ140 which lacks the first 140 amino acids to generate PDE7A;141-483.

| | |
|---|---|
| F1 | |
| CCGGGGATCCAATGGACAAGCCAAGTGTATGCT | (SEQ ID N^{o} 26) |
| H2 | |
| CGCGTCTAGATTATGATAACCGATTTTCCTGAGG | (SEQ ID N^{o} 20) |

The following primers were used to make the construct PDE7Δ170 which lacks the first 170 amino acids to generate PDE7A;171-483.

| | | |
|---|---|---|
| G1 | CCGGGGATCCATGGTAAGCTTAACCTTTCATTTA | (SEQ ID |
| | | N^{o} 27) |
| H2 | CGCGTCTAGATTATGATAACCGATTTTCCTGAGG | (SEQ ID |
| | | N^{o}20) |

Carboxy Terminal Deletions

Sequential deletions of the carboxy terminal region of PDE7A were made using PDE7Δ100 as template.

The construct PDE7; 101-450 was made using the following primers.

| | | |
|---|---|---|
| D1 | CCGGGGATCCGTGTCTGTCTCTGCAAGGAATATCAGA | (SEQ ID |
| | | N^{o} 24) |
| I2 | CGCGTCTAGATTAGCTGGCTTTATTCAGCC | (SEQ ID |
| | | N^{o} 28) |

The construct PDE7;101-430 was made using the following primers.

| | | |
|---|---|---|
| D1 | CCGGGGATCCGTGTCTGTCTCTGCAAGGAATATCAGA | (SEQ ID |
| | | N^{o} 24) |
| J2 | CGCGTCTAGATTACCTGGCCCATTCTGTAAATAAA | (SEQ ID |
| | | N^{o} 29) |

The construct PDE7;101-420 was made using the following primers. (numbers referring to the full length PDE7A1 enzyme sequence).

| | | |
|---|---|---|
| D1 | CCGGGGATCCGTGTCTGTCTCTGCAAGGAATATCAGA | (SEQ ID |
| | | N^{o} 24) |
| K2 | CGCGTCTAGATTATAGGTAAGTCATAAAACCAATCT | (SEQ ID |
| | | N^{o} 30) |

CHO Cell Culture

Chinese hamster ovary cells (EcRCHO) expressing the dimeric ecdysone receptor (Invitrogen Inc.) were cultured at 37° C. in an atmosphere of 5% carbon dioxide/95% air in complete growth medium containing Ham's F-12 nutrient mixture (Gibco BRL, Gaithersburg, Md.) supplemented with 146 mg/l L-glutamine, 10% (v/v) fetal calf serum (FCS), mM sodium pyruvate, 0.1 mM hypoxanthine, 0.4 μM aminopterine, 16 μM thymidine and 100 μg/ml gentamicin. The medium was replaced every 3-5 days, and the cells were split 1:2 once a week following trypsinization.

Transfection Assays and Preparation of Cell Extracts

Three days before transfection, CHO cells were plated at a density of 2×10⁶ cells per 150 cm² flask. Cells were transfected with 20 μg of endotoxin free plasmid DNA using Lipofectamine following the manufacturers instructions (Gibco BRL, Gaithersburg, Md.). After incubation for 48 hours, cells were washed twice with phosphate buffered saline (PBS), trypsinized, harvested by centrifugation and resuspended in 300 μL of PDE extraction buffer (Bis Tris 20 mM (pH 6.5), EDTA 10 mM, DTT 2.5 M) supplemented with a mixture of protease inhibitors at a final concentration of 2 mM benzamidine, 2 μg/ml soybean trypsin inhibitor, 50 μM PMSF and 100 μg/ml bacitracin. Cells were sonicated, centrifuged for 20 minutes at 14,000 g and the resultant supernatants were analyzed for cAMP phosphodiesterase activity.

Molt4 Cell Culture and Preparation of Cell Extracts

Molt4 were cultured IN RPMI 1640 Medium glutamine, supplemented with, 10%(v/v) fetal bovine serum (Gibco), 1 mM sodium pyruvate and 100 μg/ml gentamicin at 37° C. Cultured cells (4×108) were centrifuged and washed once with cold phosphate buffer saline. The cells were then resuspended in 300 μL of PDE extraction buffer (Bis Tris 20 mM (pH 6.5), EDTA 10 mM, DTT 2.5 μM) supplemented with a mixture of protease inhibitors at a final concentration of 2 mM benzamidine, 2 μg/ml soybean trypsin inhibitor, 50 μM PMSF and 100 μg/ml bacitracin. Cells were sonicated, centrifuged for 60 minutes at 30,000 g at 4° C. The resultant supernatants were analyzed by HPLC.

Ion Exchange HPLC Analysis

The obtained supernatant was filtered through a cellulose acetate filter (0.45 micron pore size). Approximately 1 ml were loaded onto Mono-Q ion exchange column HR 5/5 (pharmacia) previously equilibrated with buffer A (Bis Tris 20 mM (pH 6.5), EDTA 10 mM, DTT 2.5 M) supplemented with a mixture of protease inhibitors at a final concentration of 2 mM benzamidine, 2 μg/ml soybean trypsin inhibitor, 50 μM PMSF and 100 μg/ml bacitracin. After washing with buffer A, PDE activities were eluted with linear gradient of 0 M-1,0 M sodium acatate in buffer A at flow rate 0,5 ml/min. During the chromatography, the column and the eluted fractions were maintained at 4° C. 1.0 ml fractions were collected and stocked at −80° C. to be analyzed for cAMP phosphodiesterase activity.

Sf21 Cell Culture, Infection Assay

Sf21 cells were cotransfected with the expression vector and linear AcMNPV DNA (parent virus, from Invitrogen). Cell culture, recombinant virus purification and titration of the viruses were performed according the manufacturers instructions (Gibco BRL, Gaithersburg, Md.; Invitrogen, San Diego, Calif.). For protein expression, cells at a density of 2×10⁶/ml were infected with the recombinant virus at an MOI of 5. Three days post infection, cells were pelleted by centrifugation and harvested in homogenization buffer (Bis Tris 20 mM (pH 6.5), EDTA 10 mM, DTT 2.5 M) supplemented with a mixture of protease inhibitors at a final concentration of 2 mM benzamidine, 2 g/ml soybean trypsin inhibitor, 50 μM PMSF and 100 μg/ml bacitracin. Glycerol was added to a final concentration of 20-30% to all homogenates that were then stored at −20° C. in aliquots.

PDE Assays

Phosphodiesterase activities were assayed using a modification of the method described by Smith and collaborators (Manganellio et al., 1991) using the observation that 5′-nucleotides bind very strongly to aluma while the cyclic nucleotides are readily eluted at neutral or slightly alkaline pH. Assays were performed in 96 well plates at 37° C. in a total volume of 200 μl. Each well contained assay buffer (final concentration of 40 mM Tris-HCL; pH 8; 0,5 mM MgCl₂; 4 mM β-mercaptoethanol; 10 nM [³H] cAMP (Amersham) and 1 μM cAMP) and 10 μl of CHO lysate supernatant diluted dependent on the activity of the construct. Following 60 min incubation, the reaction was terminated by the addition of 1 μM [¹⁴c]AMP in TFA (0.5%). Membrane-bottomed microtiter plates (Silent monitor, Nalgen Nunc) filled with 50 mg alumina were equilibrated with 0.1M TES-NaOH, pH 8.0. The reaction mixtures were then applied to these columns. The non-hydrolysed cAMP was eluted with 3 ml equilibration buffer. The [³H] cAMP and [¹⁴c]AMP were then eluted with 2 mM NaOH directly into scintillation vials containing alkali-compatible scintillant (Ultima gold, Packard). The separated [³H] cAMP was back-corrected for recovery using the separated and non-separated [¹⁴C] values and expressed as a fraction of the total [³H] cAMP to give the amount of the substrate hydrolyzed.

Kinetics and Inhibitors Studies

PDE Assay for IC50 Measurements

The cell lysates containing PDE7A1 (full length or the truncated forms) were used as enzyme source. PDE assays were performed in 96-well plates in a final volume of 100 L at pH 8 comprising: 2 μL of compound dissolved in DMSO at 8 different concentrations, 40 mM TRIS, 10 mM MgCl₂, 50 nM [³H]-cAMP (Amersham) and enzyme at the appropriate concentration. Plates were incubated for 10 min at 37° C. in a heater/shaker and stopped by addition of 20 μL Yttrium silicate beads (Scintillation Proximity Assay, Amersham). Enzyme activity was read on a Topcount reader (Packard). Substrate depletion never exceeded 15% of the initial amount. The given IC50s represent the concentration of compound leading to 50% inhibition of the cAMP hydrolysis versus control. They are calculated using a classical sigmoidal model with Hill slope fixed to 1.

PDE Assay for Km Measurements

The cell lysates containing PDE7A1 (full length or the truncated forms) were used as enzyme source. PDE assays were performed in 96-well plates in a final volume of 100 μL at pH 8 comprising: 10 μM rolipram, 10 M of PDE3 inhibitor, 40 mM TRIS, 10 mM MgCl₂, substrate [³H]-cAMP and enzyme at the appropriate concentration. Substrate concentrations spanned a 15 nM-750 nM range. Plates were incubated for 10 min at 37° C. in a heater/shaker and stopped by addition of 20 μL Yttrium silicate beads (Scintillation Proximity Assay, Amersham). Enzyme activity was read on a Topcount reader (Packard). Substrate depletion never exceeded 15% of the initial amount. Saturation curves were then fitted with a classical sigmoidal model, Hill slope fixed to 1.

Western Blot Analysis

Lysates of CHO cells transfected with the His tagged PDE7A1 or the tagged truncated versions of PDE7A1 were frozen in liquid nitrogen, thawed three times and then centrifuged at 15,000 g for 10 minutes to remove cell debris and nuclei. The supernatant was then centrifuged at 100,000 g for 1 hour in order to isolate the plasma membrane fraction (pellet) from the cytosol fraction (the high speed supernatant).

Proteins (10 μg) were resuspended in Laemmli buffer and denatured by boiling for 5 minutes. Samples were run on a 4-15% linear gradient Tris-HCl polyacrylamide gel (100 mA/gel) and transferred to nitrocellulose membrane for detection of the fusion protein by western blotting. Membranes were blocked with 5%(w/v) low fat milk in phosphate buffered saline (PBS) Tween 20 (0.1%) for 1 hour. The blots were probed for 1 hour with the monoclonal antibody anti-his (InVitrogen) (diluted at 1 μg/ml in PBS containing 1% non fat milk and 0.1% Tween 20) which is specifically directed against amino terminus Xpress tag epitope. After washing, bound antibody was detected using anti-mouse IgG peroxidase conjugated secondary antibody (Sigma) and enhanced chemiluminescence (ECL plus, Amersham, Arlington Heights, Ill.) detection system.

Statistical Analysis

PDE activity determinations are expressed as mean +/−SEM. The non parametric Mann Whitney test was used throughout.

B. Results

### Example 1

### CHO and Baculovirus Expression of Full Length PDE7A1

The rate of cyclic AMP hydrolysis was measured in CHO cell cytosol extract, 48 hours after transfection with the recombinant full length PDE7A1 and compared to that found in non transfected cells. Cells were transfected with recombinant PDE4B2 to act as a comparison.

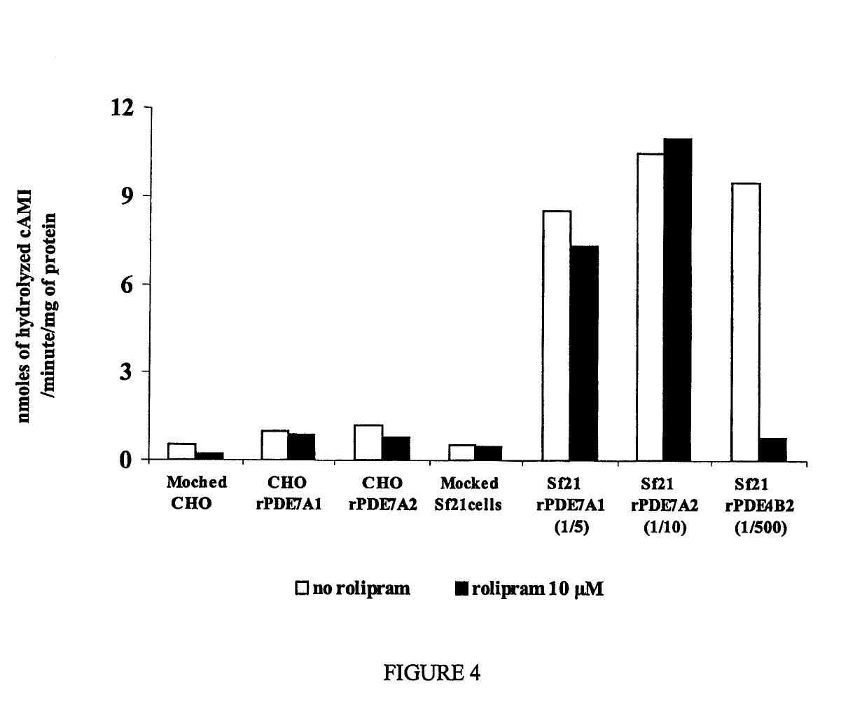

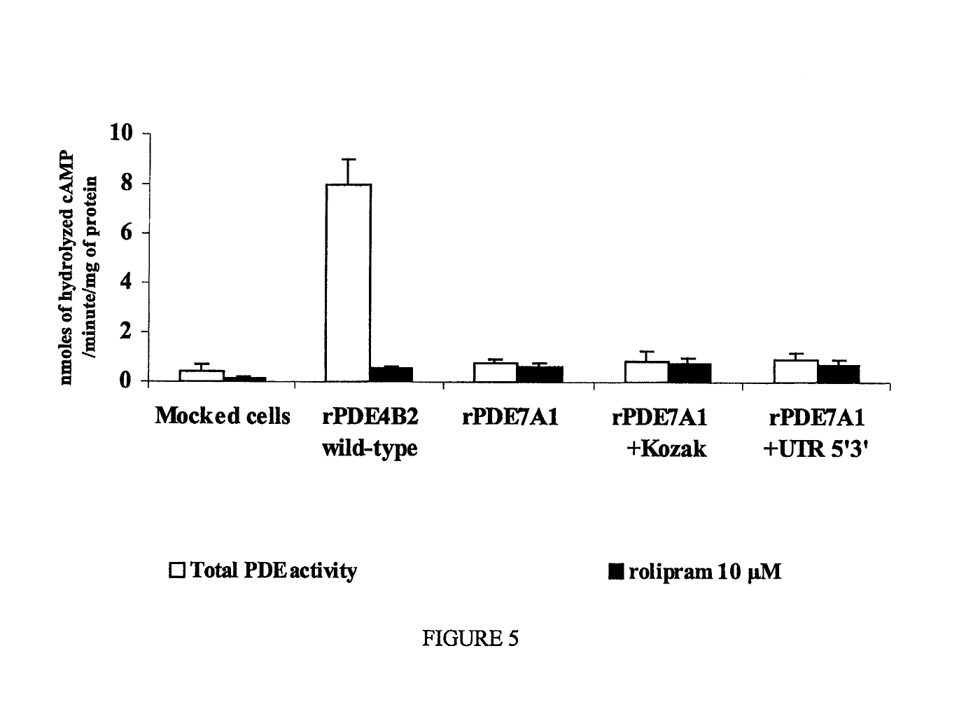

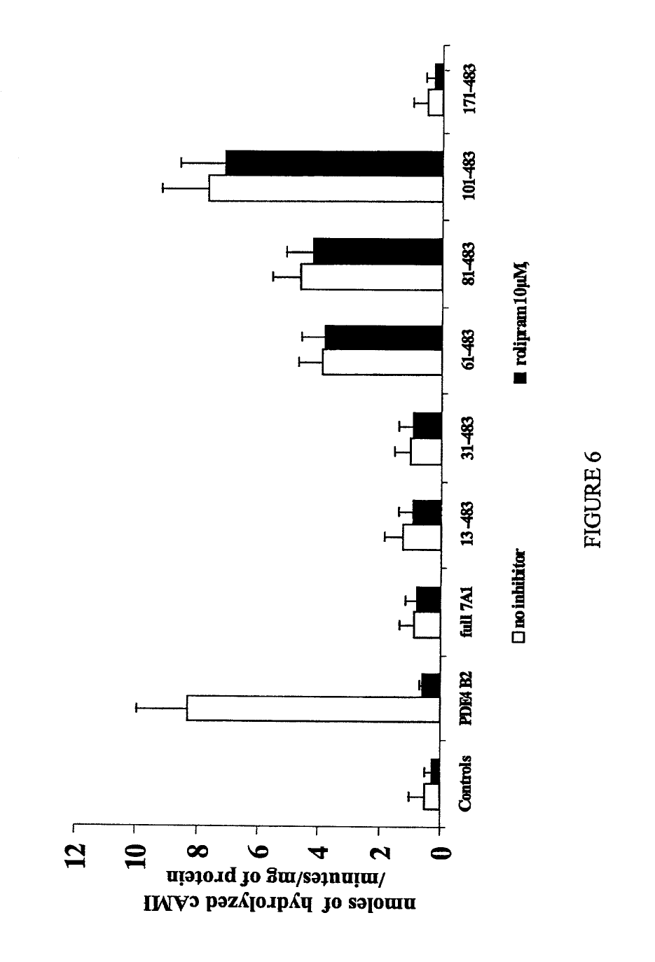

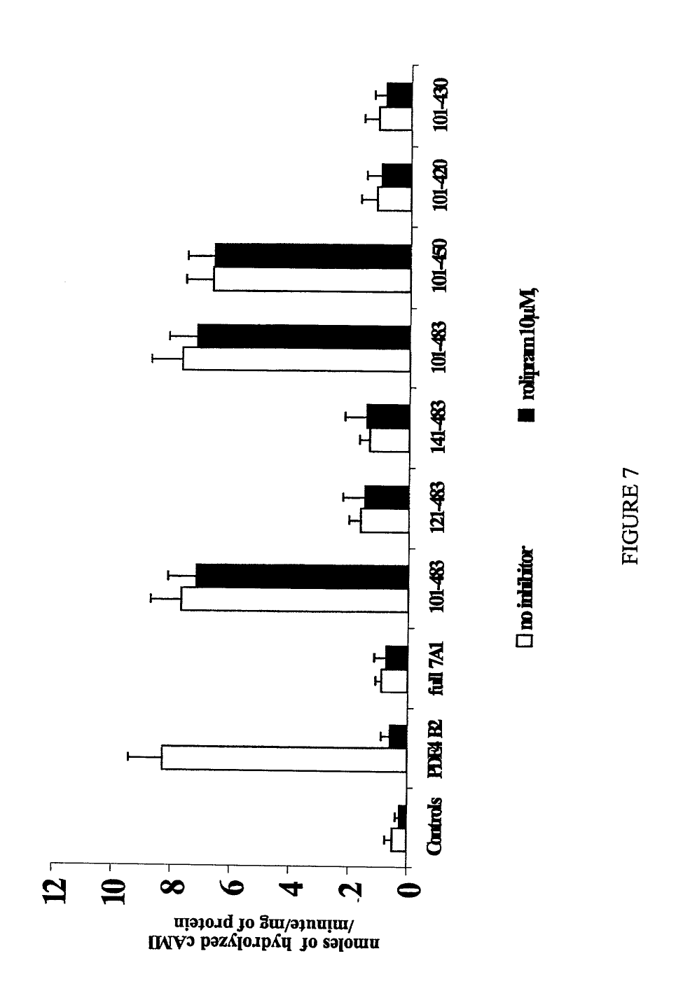

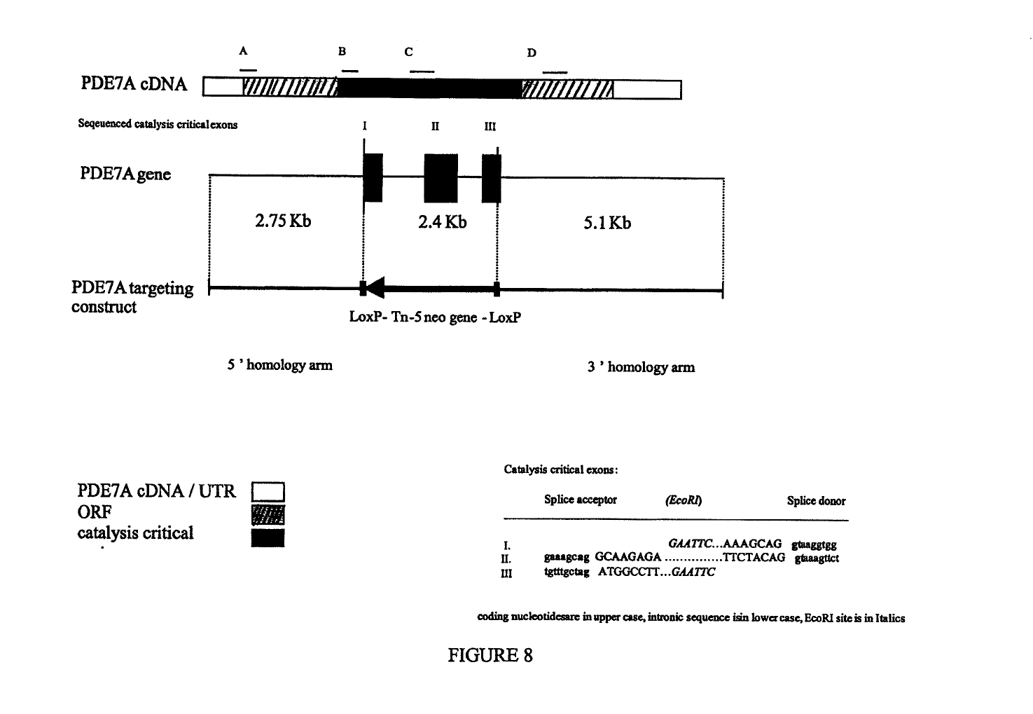

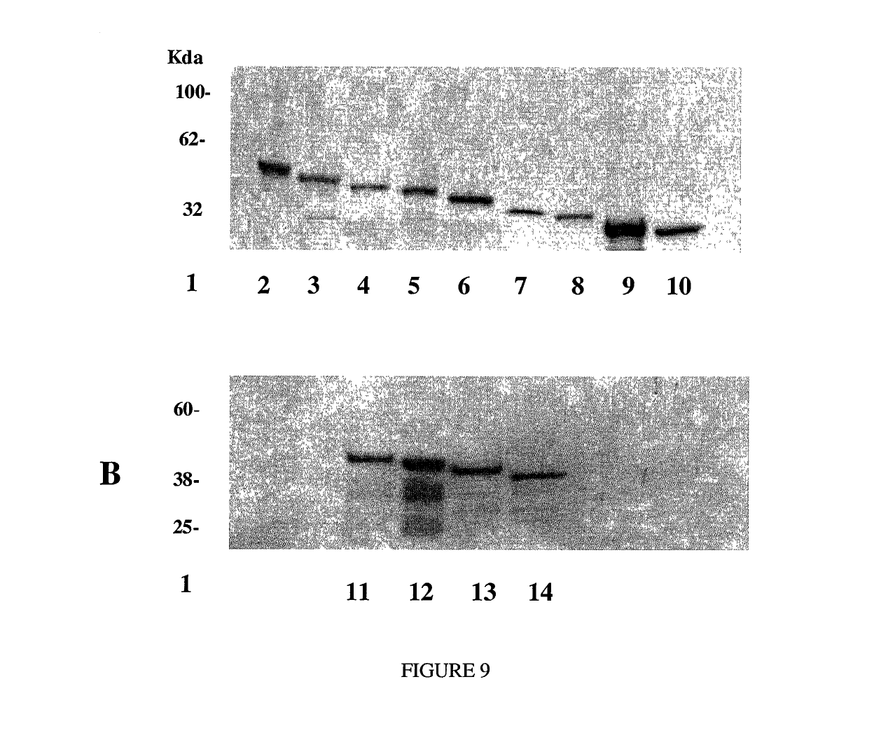

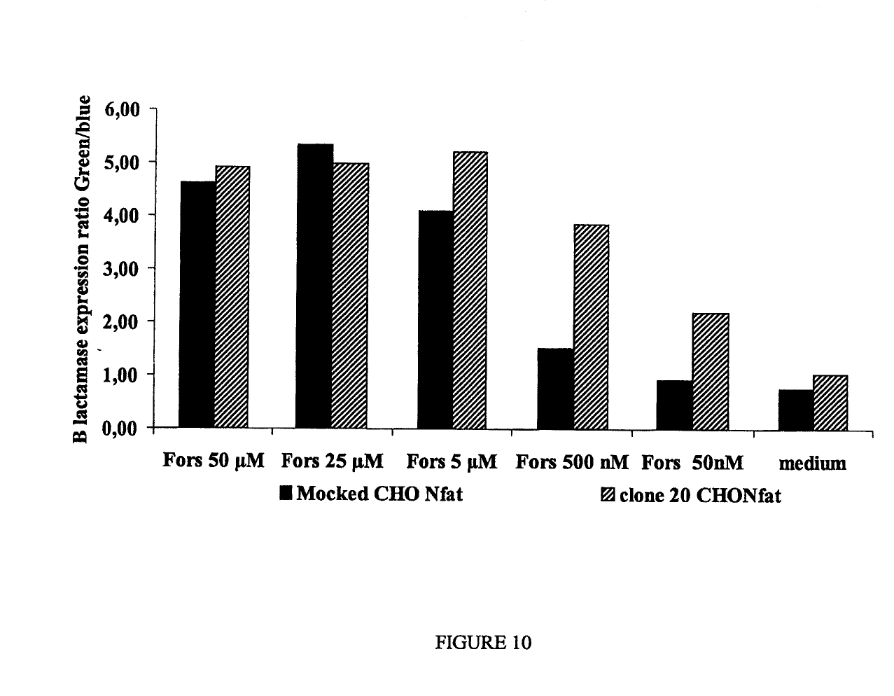

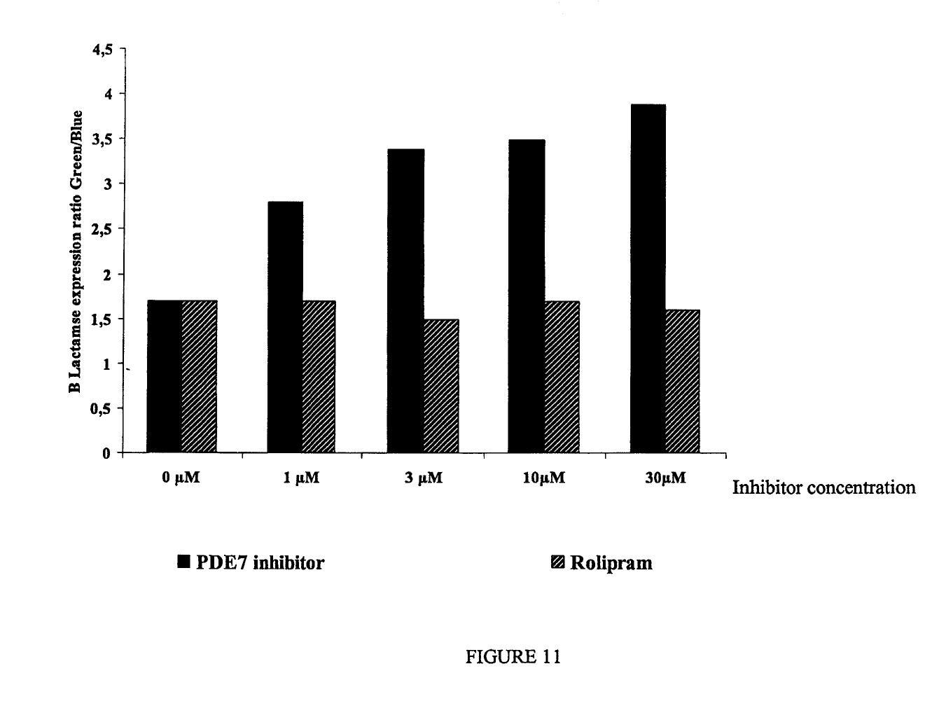

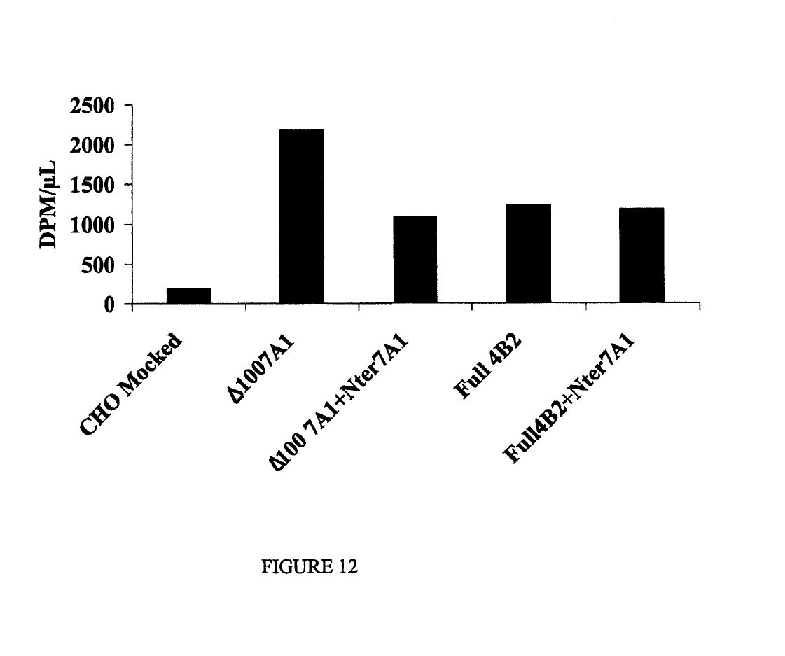

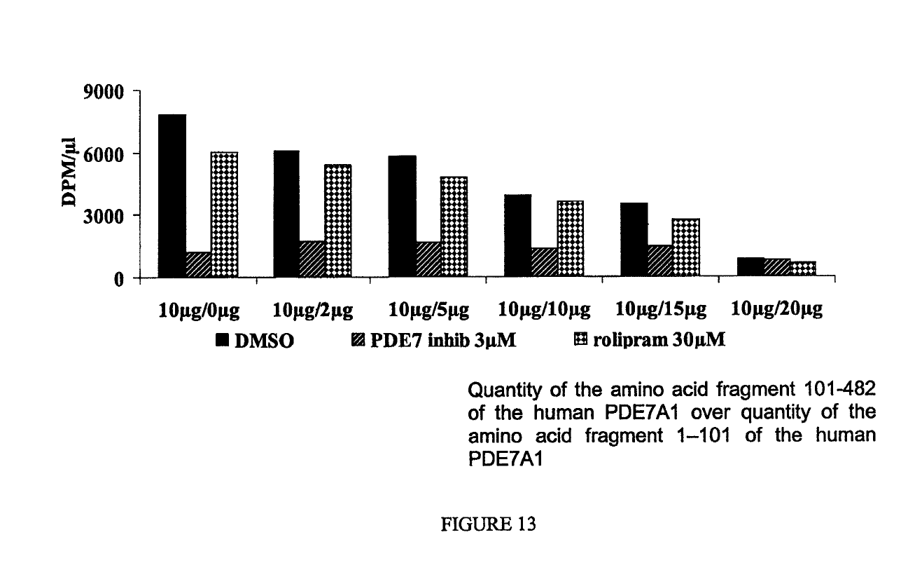

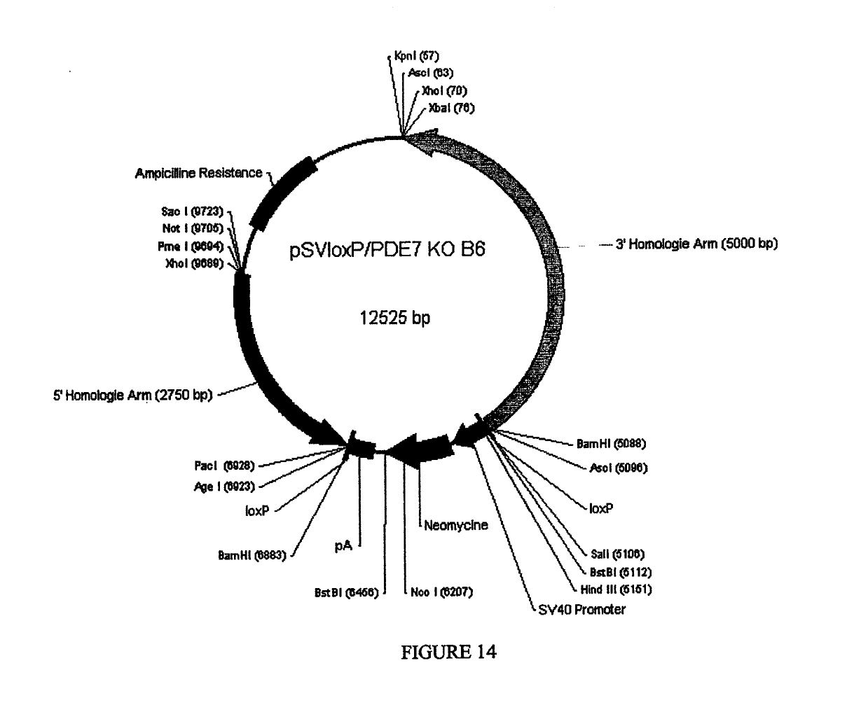

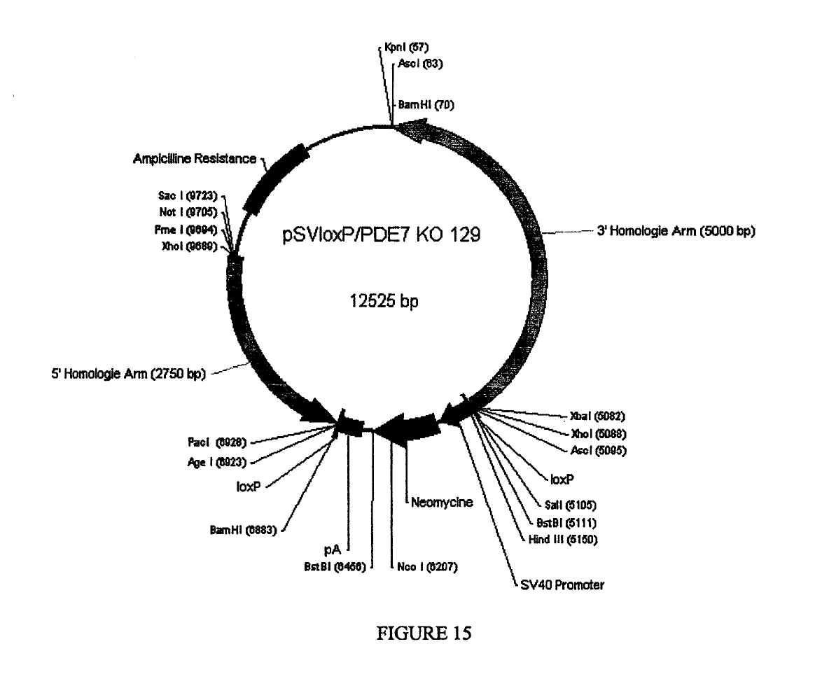

Very low levels of PDE7A activity were observed in the cytosol of cells transfected with the full length PDE7A1 compared to that observed for recombinant PDE4B2 (FIG. 4).

FIG. 4 illustrates results of PDE7A1 expression by recombinant CHO cell lines and Sf21 recombinant cell lines. The eventual dilution is mentioned between brackets.

Cyclic AMP activity was about 2-fold elevated in CHO cells expressing recombinant PDE7A1 compared to control cells. In comparison, cAMP activity was 17 fold higher for cells transfected with recombinant PDE4B2 (FIG. 5). The level of each recombinant protein detected by western blot was similar. The increase in cAMP activity above basal in the cells transfected with PDE7A1 was insensitive to rolipram (10 μM) whereas that for PDE4B2 was sensitive to rolipram (FIG. 4). These data confirm that active PDE7A1 was expressed in CHO cells but at a very low level. Similar data were obtained with PDE7A2.

In order to optimize translation, an additional construct was made to introduce an optimal Kozak sequence. In addition, the 3′ and 5′ UTR sequences were added to the PDE7A1 coding sequence in an attempt to optimize the stability of RNA of recombinant PDE7. Transient expression of these 2 additional constructs in CHO cells did not increase the level of PDE7 activity measured in the cell cytosol (FIG. 5).

Full length PDE7A1 was subcloned into an N-terminal tag baculovirus expression vector (pBlueBacHisA) and transfected into Sf21 to produce recombinant virus. Three days post-infection, Sf21 cells infected with PDE7A1 recombinant virus were assayed for PDE activity and compared to that found in uninfected Sf21 cell homogenates or cells infected with recombinant PDE4B2. PDE activity was increased in lysates from cells infected with the PDE7A1 construct compared to uninfected cells. However, consistent with the results obtained in CHO cells, the level of PDE activity in Sf21 cells expressing recombinant PDE7A1 was very low compared to levels measured in cells expressing recombinant PDE4B2 (FIG. 4). Sf21 cell homogenates infected with recombinant PDE7A1 virus had about 100-fold less cAMP activity than those infected with recombinant PDE4B2 virus. Sf21 cell homogenates infected with recombinant PDE7A2 virus had about 50-fold less cAMP activity than those infected with recombinant PDE4B2 virus.

### Example 2A

### Expression and PDE Activity of the Amino Terminal Deletions

In view of the very low levels of PDE activity obtained after transient expression of the full length coding region of PDE7A1 constructs in CHO cells various amino terminal deletions were made and these were then expressed in CHO cells. The resultant PDE activity was measured and compared to that for the full length coding region of PDE7A1 and non-transfected cells. Highest levels of PDE activity were observed for cells expressing PDE7Δ100 (FIG. 6). Thus, this clone which comprises PDE7; 101-483 exhibited a 15 fold increase in PDE catalytic activity compared to cells expressing the full length open reading frame, 1-483 and 8 fold compared to mock transfected cells. The PDE activity of this truncated PDE7 construct remained insensitive to rolipram at 10 μM and is similar to the recombinant PDE4B2 activity obtained in the same transfection conditions. The first 100 amino acids seem to have an influence on the catalytic activity of the expressed PDE7 enzyme.

Expression of PDE7Δ60 and PDE7Δ80 also resulted in an increase in PDE activity compared to expression of the full length cDNA. The increase in activity for PDE7Δ60 and PDE7Δ80 over endogenous full length PDE7Δ1 was respectively 8 and 9 fold (FIG. 6). Expression of the shorter truncations, PDE7Δ12 and PDE7Δ30, resulted in low levels of PDE7 activity that were equivalent to those observed for the full length clone. Taken together, these results indicate that the region of PDE7A that lies between residues 31 and 100 has an influence on the catalytic activity of the expressed PDE7 enzyme.

Additional amino terminal deletions of PDE7A were made. Expression of these constructs, PDE7A120, PDE7A140 and PDE7A170, resulted in a loss of PDE7 activity in cell cytosol (FIGS. 6 and 7). This suggests that the start of the catalytic domain of PDE7A lies between residues 81 and 120 (FIG. 2).

The amino terminal truncated versions of PDE7A were all expressed to equivalent levels in CHO cells as assessed by Western blot analysis using an antibody that recognized the histidine tag of these fusion proteins (FIG. 9).

FIG. 9 represents a Western blot analysis of truncated forms of recombinant human PDE7A expression in CHO transfected cells. Supernatant fractions of lysates of CHO transfected with Xpress tagged full length and N terminal truncated forms recombinant human PDE7A were prepared as described herein. Protein lysate (10 μg) were run on a 4-15% linear gradient Tris-HCL polyacrylamide gel and transferred to nitrocellulose membrane. Blot was probe with anti-Xpress monoclonal antibody directed against the NH2 terminus epitope.

FIG. 9(A): the lanes are (1) molecular mass markers, (2) Met1-483, (3) Met13-483, (4) Met31-483, (5) Met61-483, (6) Met81-483, (7) Met101-483, (8) Met121-483, (9) Met141-483, (10) Met171-483.

FIG. 9(B): the lanes are (1) molecular mass markers, (11) Met101-483, (12) Met101-450, (13) Met101-430, (14) Met101-420.

A single immunoreactive band was observed between 60 Kda and 35 Kda that was appropriate for each of the truncations. These data clearly demonstrate that all the mutant PDE7A1 proteins are expressed in CHO cells and only very minor variations in protein expression were observed between the different mutant constructions.

### Example 2B

### Confirmation of the Regulatory Role of the First 100 N-Terminal Amino Acids

We have shown above that the first 100 amino acids had an influence on the catalytic activity of the expressed PDE7 gene. This part of the protein has been proposed to act as an auto-inhibitory domain. To establish if overexpression of the first 100 amino acids can inhibit PDE7 activity of the catalytic site, this catalytic fragment (101-482) fragment was co-expressed with the fragment 1-101 of a separate plasmid or with the plasmid pcDNA3 without this insert (negative control). Cells were transfected with 15 μg of each plasmid DNA. The catalytic site activity was inhibited of only around 40-50% in presence of fragment 1-101. (FIG. 12) The effect dose response has been checked to evaluate the involvement of the first 100 amino in PDE7 activity of the catalytic domain.

A dose effect curve has been done in the same coexpression conditions. 15 μg of the catalytic fragment (101-482) was coexpressed with increasing quantity of the fragment 1-101 of a separate plasmid. The DNA quantity was normalized for each transfection using an empty vector. (FIG. 13) A effect dose response of 100 N-terminal amino acids on PDE7 activity was observed (n=3). The catalytic domain activity was inhibited until 60% in presence of the fragment 1-100 at the highest concentration.

This results seem to confirm the regulatory role of the first 100 N-terminal amino acids of the human PDE7A1.

### Example 3

### Expression and PDE Activity of the Carboxy Terminal Deletions—Mapping of the Catalytic Domain of PDE7A

Having defined the region of the start of the catalytic domain of PDE7A, three carboxy terminal deletions in the enzyme PDE7A100 were constructed to map the catalytic region of PDE7A. Expression of PDE7A;101-450 resulted in PDE activity that was equivalent to that observed for PDE7Δ100 (PDE7;101-483) suggesting that the carboxy terminal 33 amino acids play no role either in catalysis or in regulating enzyme activity (FIG. 7). However, expression of PDE7A;101-420 and PDE7A;101-430 resulted in no detectable increase in the PDE7 activity in cell extracts compared to the full length PDE7A, despite easily detectable levels of a fusion protein of the appropriate molecular weight (FIG. 7).

These results strongly indicate that the carboxy 33 amino acids part of PDE7 (451-483) are not directly involved in regulating the enzymatic activity. However the catalytic site appears to end between amino acids, 431 and 450. Thus, the PDE7 catalytic domain defined in this study between amino acids 101 and 450 has a calculated molecular mass of 42 Kda which agrees well with that estimated using SDS-polyacrylamide gel electrophoresis (40.5 kda). These results don't predict a glycosysation of the PDE7 protein.

### Example 4

### Kinetics Studies

By performing kinetics studies and Km measurements as described above, the respective Km values of the full length PDE7A1 and the truncated 101-450 PDE7A1 recombinant proteins expressed in CHO cells have been determined and are shown in Table 1 below.

TABLE 1
Km values of the full length and of the truncated 101-450
PDE7A1 proteins.

| | | truncated PDE7A1 | | |
|---|---|---|---|---|
| | Full Length PDE7A1 | polypeptide | | |
| | (from CHO cells) | 101-450 | 101-483 | Molt 4 |
| Km (nM) | 106 | 74 | 52 | 52 |
| +/− SD* | 15 | 19 | 4 | 12 |
| n* | 4 | 5 | 5 | 5 |

*Standard deviation of the mean
**number of experiments performed.

The Km for cAMP of the PDE7₍₁₀₁₋₄₅₀₎, PDE7₍₁₀₁₋₄₈₃₎, have been measured and compared to those of endogenously expressed PDE7 in human lymphocyte cell line (Molt4) and the full length wild type recombinant PDE7A1 (Table 1).

The PDE7₍₁₀₁₋₄₅₀₎, PDE7₍₁₀₁₋₄₈₃₎ demonstrated a 1.5 to 2 fold increase in catalytic efficiency compared to the recombinant full length.

Both the PDE7₍₁₀₁₋₄₅₀₎ and full length PDE7A produced in Baculovirus or eucaryote were insensitive to the PDE1 inhibitor (Vinpocetin), the PDE4 inhibitor (rolipram), the PDE3 inhibitor (SKF-94836) or the PDE5 inhibitor (Zaprinast).

Moreover, the PDE7₍₁₀₁₋₄₅₀₎, PDE7₍₁₀₁₋₄₈₃₎ and the recombinant full length PDE7A1 demonstrate similar IC50 (13-14 μM) values for the inhibitor, 3 isobutyl-1-methyl xanthine (IBMX) indicating that from the inhibitor point of view, the role of the N terminal peptide is weak.

### Example 5

### Screening for Compounds Inhibiting the PDE7 Phosphodiesterase Activity.

A. Materials and Methods

The cell lysates containing recombinant PDE7A1 (full length or the truncated forms) were used as enzyme source. PDE assays were performed in 96-well plates in a final volume of 100 μL at pH 8 comprising: 2 L of compound dissolved in DMSO at 8 different concentrations, 40 mM TRIS, 10 mM MgCl₂, 50 nM [³H]-cAMP and enzyme at the appropriate concentration.

Plates were incubated for 10 min at 37° C. in a heater/shaker and stopped by addition of 20 μL Yttrium silicate beads (Scintillation Proximity Assay, Amersham).

Enzyme activity was determined by measuring the radioactivity on a Topcount reader (Packard).

Substrate depletion never exceeded 15% of the initial amount. The given IC50s represent the concentration of compound leading to 50% inhibition of the cAMP hydrolysis versus control. They are calculated using a classical sigmoidal model with Hill slope

B. Results

Some truncated forms of PDE7A1, especially the N-terminally truncated constructs [101-483] and [101-450] have been shown to be about 15 times more active than the full length enzyme in the previous examples.

Therefore, various candidate compounds have been assayed for their inhibiting properties on the phosphodiesterase activity of respectively the full length PDE7A1 protein (1-483) and two truncated PDE7A1 polypeptides, namely (101-483) and (101-450).

Nine candidate compounds were positively selected according to the first screening method of the invention described herein. All these compound have shown interesting IC50 values, some of them have shown very interesting IC50 values. The compounds seem to behave similarly with the truncated PDE7A1 polypeptides, namely (101-483) and (101-450) respectively and the full length PDE7A1. We conclude that from the inhibitor point of view the role of the N terminal 100 amino acid peptide is weak. Deletions between amino acids 1 and 30 did not modify PDE7 enzymatic activity indicating that these residues do not influence cAMP hydrolysis or regulation. However, successive deletions performed between amino acids 31 and 100 resulted in a progressive increase in catalytic activity, suggesting that this region exerts an inhibitory constraint on PDE7 catalysis. PDE7 activity was maximal when the first 100 amino acids were deleted. The truncated protein (101-450) has a somewhat higher cAMP hydrolysing affinity (Km=0.07 μM) as compared to the full length recombinant PDE7 (Km=0.11 μM).

### Example 6

### Second screening of PDE7 inhibitors of the invention, carried out on whole cells

Subcloning Catalytic Site Under an Inducible Promoter

To determine the IC₅₀ of PDE7 inhibitors on whole cell, a stable cell line (CHO) expressing PDE7A1 catalytic site under an inducible promoter was established. The cDNA encoding human truncated PDE7A1 (101-450) was subcloned into an eucaryote inducible vector pIND (In vitrogen). The restriction enzyme sites BamHI and XbaI were used to facilitate PDE7A catalytic site subcloning into pIND vector. This clone was named pIND7A₍₁₀₁₋₄₅₀₎.

EcrCHO Cell Culture

Chinese hamster ovary cells (EcrCHO) expressing the dimeric ecdysone receptor (Invitrogen Inc.) were cultured at 37° C. in an atmosphere of 5% carbon dioxide/95% air in complete growth medium containing Ham's F-12 nutrient mixture (Gibco BRL, Gaithersburg, Md.) supplemented with 146 mg/μL-glutamine, 10% (v/v) fetal calf serum (FCS), 1 mM sodium pyruvate, 0.1 mM hypoxanthine, 0.4 μM aminopterine, 16 μM thymidine, 100 μg/ml gentamicin and Zeocine at 50 μg/ml. The medium was replaced every 3-5 days, and the cells were split 1:2 once a week following trypsinization.

Transfection Assays and Establishment Stable Cell Line

Three days before transfection, EcrCHO cells were plated at a density of 10⁶ cells per 75 cm² flask. Cells were transfected with 10 μg of endotoxin free plasmid DNA using Lipofectamine following the manufacturers instructions (Gibco BRL, Gaithersburg, Md.). After incubation for 48 hours, cells are trypsinized, harvested by centrifugation, resuspended in complete growth medium supplemented with selection antibiotic Geneticin (Gibco BRL, Gaithersburg, Md.) for pressure selection and are splited in 10 Petri dishes. After one week, Geneticin resistant clones are isolated. PDE activity is measured for each one following Ponasterone induction (10 M, during 8 h). Clones showing a PDE7 catalytic activity higher than the non transfected cells are selected to test the effect of PDE7 inhibitors on cAMP level in intact cells.

CHO cells expressing catalytic site PDE7 were seeded in 24-wells plate at a concentration of 1.5×10⁶ cells/ml culture medium. The plate is incubated for 48 h at 37° C. in an humidified atmosphere of 95% air-5% CO₂. After 36 h, Ponasterone (10 μM) is added in the culture medium for 8 hours induction period. After the induction time, the culture medium is removed by aspiration and each well is washed with PBS. The candidate molecules are added to the well diluted in 150 mM Tris/HCl buffer, 2 mM EDTA, pH 7.4 to reach the desired concentrations to test. The cells are then incubated for 30 min at 37° C. under gentle shaking before addition of 10 M forskolin, adenylate cyclase activator, for 15 min. After the activation time, the plate is stored on ice and 200 μl of stop solution, composed by 0.2M HCl, 1% Triton X100, 0.2 mM IBMX, is added in each well. The cells are then disrupted by sonication to allow the measurement of intracellular cAMP produced during the reaction.

cAMP Measurement

cAMP levels in each cell population treated with candidate molecule were evaluated using an EIA kit from Euromedex. This kit is a competitive immunoassay for the quantitative determination of cAMP in samples treated with 0.1 M HCl. The kit uses a polyclonal antibody to cAMP to bind, in a competitive manner, the cAMP in the sample or an alkaline phosphatase molecule which has cAMP covalently attached to it. 280 μl of each sample are transferred to 96-wells plate to separate cellular cytosol from particulate fraction by centrifugation, 15 min at 3500 rpm, 4° C. The cytosolic fraction separated from the pellet is then transferred to a “transfer 96-wells plate”, which is stored on ice until protein and cAMP measurement. 195 μl of each sample along with samples from the cAMP standard curve provided in the kit, are acetylated with 10 μl of a mix of 1:½ triethylamine: anhydrous acetic acid before competitive cAMP-EIA.

### Example 6B

### Optimization of the Second Screening of PDE7 Inhibitors Second Screening of PDE7 Inhibitors on Stable Cell Line Coexpressing PDE7Catalytic Site and Gene Reporter.

In order to set up a cellular assay to test PDE7 inhibitors on whole cells, a stable cell line (EcrCHO) co-expressing PDE7 catalytic domain (active form) and a reporter gene β lactamase under a CRE4X promoter has been initiated. Integration of a reporter gene under a CRE4X promoter should have to optimize the measurement of cAMP in cell based assays Previous studies showed that overexpression of PDE is lethal for the cells, so cDNA of the PDE7 (101-450) catalytic site was cloned in the pIND vector for inducible expression in mammalian cells.

Subcloning Catalytic Site Under an Inducible Promoter

The cDNA encoding human truncated PDE7A1 (101-450) was subcloned into an eucaryote inducible vector pIND (In vitrogen). The restriction enzyme sites BamHI and XbaI were used to facilitate PDE7A catalytic site subcloning into pIND vector. This clone was named pIND7A(101-450). The new construct has been obtained by RT-PCR, the absence of mutation has been checked by sequencing.

EcrCHO Cell Culture

Chinese hamster ovary cells (EcrCHO) expressing the dimeric ecdysone receptor (Invitrogen Inc.) were cultured at 37° C. in an atmosphere of 5% carbon dioxide/95% air in complete growth medium containing Ham's F-12 nutrient mixture (Gibco BRL, Gaithersburg, Md.) supplemented with 146 mg/l L-glutamine, 10% (v/v) fetal calf serum (FCS), 1 mM sodium pyruvate, 0.1 mM hypoxanthine, 0.4 μM aminopterine, 16 M thymidine, 100 μg/ml gentamicin and Zeocin (In vitrogen cat R250-05) at 250 μg/ml. The medium was replaced every 3-5 days, and the cells were split 1:2 once a week following trypsinization.

Transfection Assays and Establishment Stable Cell Line

Three days before transfection, EcrCHO cells were plated at a density of 10⁶ cells per 75 cm² flask. PDE7 (101-450) in pIND (hygromycin selection) and β lactamase gene (neomycin selection) have been cotransfected in EcrCHO cells (zeocine selection).

Cells were cotransfected with 10 μg of endotoxin free plasmid using Lipofectamine following the manufacturers instructions (Gibco BRL, Gaithersburg, Md.). After incubation for 48 hours, cells were cultived in complete growth medium supplemented with selection antibiotic Geneticin 800 μg/ml (Gibco BRL, Gaithersburg, Md. ref 10131-027) and hygromycin B 300 μg/ml (Clontech cat:8067-1). After 2 weeks, Geneticin and Hygromycin resistant clones are treated with Forskoline at 10 μM during 4 hours. Cells expressing beta lactamase gene are isolated in 96 wells using Facs.

After 3 weeks, clones geneticin and hygromycin resistant are selected. PDE activity is measured for each one following Ponasterone A ( Invitrogen cat H101-01) induction (10 μM, during 8 h). Clones showing a PDE7 catalytic activity higher than the non transfected cells are selected to determine the level expression of gene reporter in each one in order to select the best to test the effect of PDE7 inhibitors in intact cells. cells were washed twice with phosphate buffered saline (PBS), trypsinized, harvested by centrifugation and resuspended in 300 μL of PDE extraction buffer (Bis Tris 20 mM (pH 6.5), EDTA 10 mM, DTT 2.5 M) supplemented with a mixture of protease inhibitors at a final concentration of 2 mM benzamidine, 2 μg/ml soybean trypsin inhibitor, 50 μM PMSF and 100 μg/ml bacitracin. Cells were sonicated, centrifuged for 20 minutes at 14,000 g and the resultant supernatants were analyzed for cAMP phosphodiesterase activity.

Phosphodiesterase Assays

PDE activities were assayed using a modification of the method described by Smith and collaborators using the observation that 5′-nucleotides bind very strongly to alumina while the cyclic nucleotides are readily eluted at neutral or slightly alkaline pH. Assays were performed in 96-well plates at 37° C. in a total volume of 200 μl. Each well contained assay buffer (final concentration of 40 mM Tris-HCL, pH 8, 0.5 mM MgCl₂, 4 mM β-mercaptoethanol, 10 nM [³H] cAMP and 1 μM cAMP) and 10 μl of CHO lysate supernatant diluted dependent on the activity of the protein expressed. Following 60 min incubation, the reaction was terminated by the addition of 1 M [¹⁴C] AMP in TFA (0.5%). Membrane-bottomed microtiter plates (Silent monitor, Nalgen Nunc) filled with 50 mg alumina were equilibrated with 0.1 M TES-NaOH, pH 8.0. The reaction mixtures were then applied to the columns. The non-hydrolysed cAMP was eluted with 3 ml equilibration buffer. The [³H] cAMP and [¹⁴C] AMP were then eluted with 2 mM NaOH directly into scintillation vials containing alkali-compatible scintillant (Ultima gold, Packard). The separated [³H] cAMP was background-corrected for recovery using the separated and non-separated [¹⁴C] values and expressed as a fraction of the total [³H] cAMP to give the amount of the substrate hydrolyzed.

Beta Lactamase Expression in EcrCH07 Cells Expressing Catalytic Site PDE7

Cells were seeded in 96-wells plate at a concentration of 1.3×10⁵ or 2.6×10⁵ cells/ml culture medium. The plate is incubated for 24h00 or for 8h00 for a concentration of 1.3×10⁵ cells/ml or for a concentration of 2.6×10⁵ cells/ml respectively at 37° C. in an humidified atmosphere of 95% air-5% CO₂. After 8h00 to 24h00, Ponasterone (10 μM 1 mM stock dissolved in 100% ethanol) is added in the culture medium for 16 hours induction period. After the induction time, the culture medium is removed by aspiration and each well is washed with PBS. The candidate molecules are added to the well diluted in growth medium containing Ham's F-12 nutrient mixture (Gibco BRL, Gaithersburg, Md.) to reach the desired concentrations to test. Compounds are tested in presence of 50 nM forskolin (sigma cat:F6886), adenylate cyclase activator. Positive control is IBMX at 500 μM in presence of Forskolin at 50 nM. Negative control is Forskolin alone at 50 nM. The cells are then incubated for 4 hours at 37° C. After the activation time, cells were loaded with substrat CCF4 (12 l) diluted of a solution solution B (60 μl) and solution C (1 ml). 20 l of this solution is added in each well. The cells are then incubated 1h30 at 25° C. screened from light. The measurement of intracellular cAMP increase produced by PDE inhibitor treatment is correlated to an induction of Beta lactamase expression.

Beta Lactamase Expression Measurement

cAMP levels in each cell population treated with candidate molecule were evaluated using the gene reporter expression Beta lactamase sensible to cAMP level in cell.

Results

Clones Selection Using Facs

10 days after transfection, cells expressing β lactamase after Forskolin treatment have been cloned using FACS. 25 clones neomycin, zeocin and hygromycin resistant have been obtained, the PDE7 activity of each one after ponasterone induction have to be measured.

PDE7 Activity in EcrCHO Cell Expressing Beta Lactamase Gene

3 clones which express PDE7 enzyme after ponasterone induction (8 h, 10 μM) have been selected. Total PDE activity is 3 to 6 higher than in the non transfected cells. The PDE7 activity has been confirmed using a PDE7 inhibitor and a PDE4 inhibitor (rolipram, 60 μM).

Expression of Reporter Gene (α Lactamase)

β lactamase expression has been evaluated for each clone following stimulation with forskolin (50 nM to 50 μM) (FIG. 10). The submaximal forskolin concentration (50 nM) was chosen to increase basal cAMP levels for testing PDEs inhibitors (FIG. 10). A concentration curve of a PDE7 inhibitor and rolipram has been performed on 3 clones at various cellular concentrations (15000 and 20 0000 cells/well; and 30000 and 40000 cells/well). FIG. 11 shows results obtained for one of the clones. Preliminary studies showed a concentration dependency for the PDE7 inhibitor on β lactamase expression but no effect of PDE4 inhibitors.

### Example 7

### Construction of a Homologous Recombination Vector of the Invention Designed for Generating PDE7 Knock-Out Transgenic Mice.

Human PDE7A1 and PDE7A2 are splice forms of the high affinity cAMP specific PDE7A enzyme. Differential splicing results in two variants of the N-terminal peptide sequence (PDE7A1: M1-Q46, PDE7A2: M1-K20) and it is the consequence of alternative coding exon usage (Bloom et al, 1996;Han et al., 1997).

No splice variants 3′ of Q46 in PDE7A1 and K20 in PDE7A2, affecting the catalytic domain of the enzyme were reported; nucleotide and peptide sequences beyond these points are identical in PDE7A1 and PDE7A2.

Therefore the catalysis critical domain of PDE7A and a downstream portion of PDE7A has been selected for targeting in mouse ES cells by homologous recombination.

Catalysis critical region of the human PDE7A has been determined in the examples above.

Construction of the Homologous Recombination Vector (Targeting Vector)

A 2.4 Kb, C57BU6 mouse EcoRI genomic fragment coding for exons internal to the critical catalytic region and highly conserved between mouse and human was cloned from an EcoRI mini-library prepared from the 133O20 BAC clone (RPCI-23/C57BU6 mouse BAC library, Pieter DeJong, Research Genetics, Roswell Park Cancer Research Institute). The probe was a radio-labelled oligonucleotide (probe C 1100-1077*- SEQ ID No 9).

Sequencing identified exon and splice site sequences on the 2.4 kb EcoRI genomic fragment as shown on FIG. 8. For the construction of the targeting vector, the 3′ neighbouring 5.1 Kb EcoRI genomic fragment has been cloned with a radiolabelled oligonucleotide probe (probe D 1267-1297*—SEQ ID No10) and partially sequenced. Sequence analysis indicates that this fragment contains all of the 3′ flanking exons.

The 5.1 Kb EcoRI fragment will serve as the 3′ homology arm in the targeting vector. To obtain the 5′ homology arm, PCR primers (upper primer-A, 494-513- SEQ ID No 7, lower primer-B 831-807*- SEQ ID No 8) were synthesised and used in PCR reactions to amplify a 2.75 Kb genomic fragment upstream of the 2.4 kb EcoRI fragment. The 2.75 Kb fragment was amplified from 133020 BAC DNA.

Similar strategy was employed to obtain the 5′ and 3′ homology arms (5,0 and 2.75 Kb respectively) from the genome of the 129 mouse strain. In these experiments the 125 B 22 BAC clone was used, which was cloned from RPCI-22 BAC Library as described for the C57BL6 strain. *numbers refer to nucleotide numbers in the mouse PDE7A2 cDNA (SEQ ID No 11).

The two homology arms were inserted into the pSV-loxP vector to generate the targeting vector as shown on FIG. 14 (C57BU6 vector) and FIG. 15 (129 vector).

The catalytic portion of the PDE7 contained in the C57BU6 vector has the sequence of SEQ ID No 35.

To test the desired targeting event in C57BU6 mice, Southern blot screening assays were developed. These assays discriminate homologous recombination of each arms from random integration of the entire vector which leaves the wild type alleles intact. To detect recombination of the 5′ homology arm, a 400 bp probe was generated by PCR, using oligos 1 and 2. Oligonucleotide sequences, 1 (GGGTTTACTGTGCTCTCCA, SEQ ID No 31) and 2 (TGCCACCACACTCTGCTCT, SEQ ID No 32) were designed based on DNA sequence upstream from the arm, which was obtained by sequencing of the BAC clone. Similarly, a 600 bp probe was generated by PCR with the following oligos, 3 (AAAAACTAACTAACTAACTAAA, SEQ ID No33) and 4 (CTTCTATACATTGGCTTGATTG, SEQ ID No34) downstream of the 3′ homology arm. These probes are expected to discriminate the wild type allelle from the recombinant in Southern blot experiments using a selected set of restriction enzymes as shown on the table 2 below.

TABLE 2
Size is expressed in Kb and the two different sizes are: WT/KO

| 5′ end PROBE: | | | |
|---|---|---|---|
| Digested | | | |
| with: | Bam HI | Bgl II | BgI I + Bgl II |
| Expected | 13.5/11.9 | 3.8/15.9 | 3.8/6.9 |
| Size in Kb | | | |
| Tested | 13.5/nd | 3.8/nd | nd |
| Size in Kb | | | |

| 3′ end PROBE: | | |
|---|---|---|
| Digested | | |
| with: | Bam HI | Bgl II |
| Expected | 13.5/13.3 | 9.3/14.9 |
| Size in Kb | | |
| Tested | 13.5/nd | 9.3/nd |
| Size in Kb | | |

Successful targeting events are expected to generate a mutant PDE7A allele which will lack peptide sequences indispensable for PDE7A enzymatic activity.

Use of the Vector to Generate Knock-Out Mice for PDE7.

The targeting vector are introduced into ES cells by electroporation or other methods. Neomycin resistant colonies are screened for the identification of specific targeting events.

In a possible variant of the experiment, transient Cre recombinase expression in ES cells are used to remove the loxP flanked Tn-5 Neomycin resistance gene from the targeted allele.

Once ES cell colonies with targeted alleles are identified, blastocysts are injected with ES cells from these colonies. Mice with high degree of ES cell contribution are screened by coat colour examination, germline transmitting mice are selected by breeding and tail DNA testing. Once hemizygous targeted mice (PDE7A −/+) are obtained, they are tested in biological experiments together with homozygous null allele (PDE7A −/−) mice (if these are viable) which are generated by breeding (see Gene targeting, 1993, Ed. A. L. Joyner IRL Press, Oxford).

In yet another experiment the 2.4 Kb genomic fragment are flanked by LoxP sites and introduced in it's genomic context between the 5′ and 3′ homology arms. Internal to the a LoxP site on one side, the Tn-5 neo cassette are also introduced. Homozygous, insertion positive mice are generated and crossed with tissue specific Cre recombinase expressing transgenic mice. The expected result of this experiment is tissue specific deletion of the PDE7A gene. If the Cre recombinase is controlled by an inducible promoter, deletion of PDE7A are inducible.

Probes and PCR primers:

| | | |
|---|---|---|
| Primer-A: | AAATGGAAATAGTCTAGTAA | (SEQ ID |
| | | N^{o}7) |
| Primer-B: | AATTCTTGTATAAAGTTGCTAGATA | (SEQ ID |
| | | N^{o}8) |
| Probe-C: | ACATGGGTTACAAATATCAGCACA | (SEQ ID |
| | | N^{o}9) |
| Probe-D: | CTTTATTTACAGAGTGGGCCAGGTTTTCAGC | (SEQ ID |
| | | N^{o}10) |

### REFERENCES

ABBONDANZO S J et al., 1993, Methods in Enzymology, Academic Press, New York, pp.803-823.

ADAMS et al., 1985, Nature 318:533-538.

AUSUBEL ET AL., 1989. CURRENT PROTOCOLS IN MOLECULAR Biology, Green Publishing Associates and Wiley Interscience, N.Y.

BAEHR, W., DEVLIN, M. J., AND APPLEBURY, M. L. (1979) J. BioL Chem. 254,11669-11677

BEAVO J A, HARDMAN J G, SUTHERLAND E W (1970) Hydrolysis of cyclic guanosine and adenosine 3′,5′-monophosphates by rat and bovine tissues. J Biol Chem 245:5649-5655

BEAVO, J. A. (1995) Physiol. Rev. 75, 725-748,

BENOIST AND CHAMBON, 1981, Nature 290:304-310.

BLOOM J. et. al. PNAS 93:14188-14192 (1996)

BRINSTER et al., 1982, Nature 296:39-42.

BRINSTER ET AL., 1985 Annu. Rev. Genet. 1986 20: 465-499

CAPECCI M et al., 1991, Science, vol.244:1288-1292.

CAWLEY P. ET AL, IN VITRO, 1977

CONTI ET AL., 1995, Endocrine Review, VOL 16 (3):370-389

CHAI H. et al., 1993, Biotechnol. Appl. Biochem, vol.18: 259-273

DEBOER, ET AL., 1983, PROC. NATL. ACAD. SCI. U.S.A. 80:21-25.

FINK T L et al., 1999, The Journal of Biological Chemistry, vol.274 (49): 34.613-34.620.

FRESHNEY (ed.), 1986. Immobilized Cells And Enzymes, IRL Press.

FUNG, B. K. -K., YOUNG, J. M., YAMANE, H. K., AND GRISWOLD-PRENNER, I. (1990) BIOCHEMISTRY 29,2657-2664

GAIT (ed.), 1984. Nucleic Acid Hybridization.

GENE TARGETING. Ed. A. L. Joyner IRL press/Oxford (1993)

GLOVER (ed.), 1985. DNA Cloning: A Practical Approach, Volumes I and II Oligonucleotide Synthesis, MRL Press, Ltd., Oxford, U.K.

GROSSCHEDL et al., 1984, Cell 38:647-658.

GU H. et al., 1993, Cell, viol.73:1155-1164

GU H. et al. 1994, Science, vol. 265:103-106.

HAMES AND HIGGINS (eds.), 1985. Transcription And Translation.

HAMES B D AND HIGGINS S J, 1985. Nucleic acid hybridization: a practical approach, Hames and Higgins Ed., IRL Press, Oxford.

HAN P. ET. AL. JBC. 272:16152-16157 (1997)

HANAHAN, 1985, Nature 315:115-122.

HOESS et al., 1986, Nucleic Acids Research, vol.14:2287-2300.

HOUBEN WEYL, 1974, In Meuthode der Organischen Chemie, E. WUNSCH Ed., vol.15-I and 15-II, THIME, Stuttgart.

ICHIMURA M. AND KASE H., 1993;

JAESEUNG LIM, GUDRUN PAHLKE, AND MARCO CONTI J. Biol. Chem. 1999 274:19677-19685), PDE1 (Beavo etal., 1995)

JIN, S. L., SWINNEN, J. V., AND CONTI, M. (1992) J. Biol. Chem. 267, 18929-18939

KAUFMAN, 1991, Current Protocols in Molecular Biology, 16.12

KELSEY ET AL., 1987

KOLLIAS et al., 1986, Cell 46:89-94.

KOVALA, T., SANWAL, B. D., AND BALL, E. H. (1997) Biochemistry 36, 2968-2976;

KRUMLAUF et al., 1985, Mol. Cell. Biol. 5:1639-1648.

LAEMMLI, U K,(1970) Nature, 55,680-685

LEDER et al., 1986, Cell 45:485-495.

LENHARD T. et al., 1996, Gene, vol.169:187-190.

MACDONALD, 1987, Hepatology 7:425-515.

MANGANELLIO ET AL.(1991) J. Biol. Chem., 266,13385-13390

MASON et al., 1986, Science 234:1372-1378.

MERRIFIELD R b, 1965, Science, vol.150(693): 178-185.

MERRIFIELD R B, 1965a, Nature, Vol.207 (996):522-523

MICHAELI et al, 1993, J. Biol. Chem., 268,12925-12932

MICHAELI ET AL., (1997), J. Biol. Chem. 268,12925-12932

MOGRAM et al., 1985, Nature 315:338-340

NEEDLEMAN AND WUNSCH (1972) J. Mol. Biol. 48:442

ORNITZ et al., 1986, Cold Spring Harbor Symp. Quant. Biol. 50:399-409.

PEARSON AND LIPMAN (1988) Proc. Natl. Acad. Sci (USA) 85:2444

PEASE S and WILLIAMS R S, 1990, Exp. Cell. Res.vol.190:209-211

PERBAL, 1984. A Practical Guide To Molecular Cloning.

Pinkert et al., 1987, Genes and Devel. 1:268-276.

READHEAD et al., 1987, Cell 48:703-712.

SAMBROOK, J. FRITSCH, E. F., and T. MANIATIS, 1989. Molecular cloning: a laboratory manual 2ed. Cold Spring Harbor Laboratory, Cold spring Harbor, N.Y.

SANI, 1985, Nature 314:283-286.

SMITH AND WATERMAN (1981) Adv. Appl. Math. 2: 483, by the homology alignment algorithm of Needleman and Wunsch (19720) J. Mol. Biol. 48:442

SMITH et al., 1988, Gene 67:31-40.

SONNENBURG W K et al., 1995, The Journal of Biological Chemistry, vol.270 (52): 30.989-31.000.

STROOP, S. D., CHARBONNEAU, H., and BEAVO, J. A. (1989) J. Biol. Chem. 264,13718-13725

SWIFT et al., 1984, Cell 38:639-646.

TORPHY T J et al. 1993, in New drugs in allergy and asthma, Birkhauser Verlag editors, Basel, Switzerland, pages 51-71).

TUCKER, M. M., ROBINSON, J. B., Jr., and STELLWAGEN, E. (1981) J. Biol. Chem. 256, 9051-9058

Useful proteins from recombinant bacteria” in Scientific American, 1980, 242:74-94

VILLA-KAMAROFF, et al., 1978, Proc. Natl. Acad. Sci. U.S.A. 75:3727-3731.

VLASAK R et al., 1983, Eur. J. Biochem., vol.135:123

WAGNER ET AL., 1981

Wu and Wu, 1987. J. Biol. Chem. 262:4429-4432.

WU AND WU, 1988, J. Biol. Chem. 263:14621-14624.

WU et al., 1992, J. Biol. Chem. 267:963-967.

YAMAMOTO, et al., 1980, Cell 22:787-797.

ZOU Y R et al., 1994, Current Biology, vol.4:1099-1103.

1. A polypeptide possessing a phosphodiesterase catalytic activity at least about 6 fold, preferably about 8 or 10 fold, most preferably 15 to 20 fold higher than the phosphodiesterase catalytic activity of an endogenous full length PDE7 protein which comprises at least the catalytic domain of the PDE7 with the exception of the amino acid sequence disclosed by Michaeli et al (1993, J. Biol. Chem., 268, 12925-12932) as the sequence consisting of the NH2 terminal deletion of HCP1 generated by sequential deletion of the sequence from the first ATG codon to residue 81 (L22M2).

2. A polypeptide according to claim 1 of up to about 427 amino acids in length possessing a phosphodiesterase 7 catalytic domain and comprising at least 312 consecutive amino acids of a sequence selected from the group consisting of the amino acid sequences of SEQ ID No 1, 2 or 3; or a homologous polypeptide thereof.

3. The polypeptide according to claim 2 which comprises the aminoacid sequence which:
begins at the aminoacid residue located in position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 of SEQ ID No1, 2 or 3; and which
ends at the aminoacid residue located in position 375, 376, 377, 378, 379, 380, 381, 382, 383, 384, 385, 386, 387, 388, 389, 390, 391, 392, 393, 934, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427 of SEQ ID No1, 2 or 3 or a homologous peptide thereof.

4. The polypeptide according to claim 3 which comprises the amino acid sequence beginning at the amino acid residue in position 5 and ending at the amino acid residue in position 427 of SEQ ID No1, 2 or 3; or a homologous polypeptide thereof.

5. The polypeptide according to claim 3 which comprises the amino acid sequence beginning at the amino acid residue in position 25 and ending at the amino acid residue in position 427 of SEQ ID No1, 2 or 3; or a homologous polypeptide thereof.

6. The polypeptide according to claim 3 which comprises the amino acid sequence beginning at the amino acid residue in position 45 and ending at the amino acid residue in position 427 of SEQ ID No1, 2 or 3; or a homologous polypeptide thereof.

7. The polypeptide according to claim 3 which comprises the amino acid sequence beginning at the amino acid residue in position 5 and ending at the amino acid residue in position 394 of SEQ ID No1, 2 or 3; or a homologous polypeptide thereof.

8. The polypeptide according to claim 3 which comprises the amino acid sequence beginning at the amino acid residue in position 25 and ending at the amino acid residue in position 394 of SEQ ID No1, 2 or 3; or a homologous polypeptide thereof.

9. The polypeptide according to claim 3 which comprises the amino acid sequence beginning at the amino acid residue in position 45 and ending at the amino acid residue in position 394 of SEQ ID No 1, 2 or 3; or a homologous polypeptide thereof.

10. A polypeptide having at least 80% homology or identity, preferably 85% homology or identity, with a polypeptide as defined in anyone of claims 1 to 9.

11. A polypeptide having at least 90% homology or identity, preferably 95% homology or identity, most preferably 99% homology or identity with a polypeptide as defined in anyone of claims 1 to 9.

12. A polypeptide comprising the amino acid sequence which begins at an amino acid comprised between amino acids 57 to 100 and ends at an amino acid comprised between amino acids 450 to 483 of the full-length PDE7(A) protein with the exception of the amino acid sequence disclosed by Michaeli et al (1993, J. Biol. Chem., 268, 12925-12932) as the sequence consisting of the NH2 terminal deletion of HCP1 generated by sequential deletion of the sequence from the first ATG codon to residue 81 (L22M2).

13. A polypeptide according to claim 12 wherein the PDE7A protein is human PDE7A1 or PDE7A2.

14. A nucleic acid sequence encoding a polypeptide as defined in anyone of claims 1 to 13, or a sequence complementary thereto.

15. The nucleic acid sequence according to claim 14 which is selected from the group consisting of the nucleic acid sequences of SEQ ID No4, 5 or 6; or a sequence complementary thereto.

16. A nucleic acid sequence comprising the portion of the sequence of the full length PDE7(A) which remains after deletion of a nucleic acid sequence according to claims 14 to 15.

17. A nucleic acid sequence according to anyone of claims 14 to 16 which is of genomic origins.

18. A nucleic acid sequence according to claim 16 or 17 wherein the deleted nucleic acid portion is replaced by a heterologous polynucleotide sequence.

19. A polypeptide according to anyone of claims 1 to 13 or a nucleic acid d sequence according to anyone of claims 14 to 18, wherein PDE7(A) is of human, mouse or rat origin, most preferably human.

20. The nucleic acid according to claim 18, wherein the heterologous polynucleotide comprises a selection marker.

21. The nucleic acid according to claim 18, wherein the heterologous polynucleotide comprises at least a loxP sequence at its 5′ end and at least a loxP sequence at its 3′ end.

22. The nucleic acid of any one of anyone of claims 14 to 21, wherein the nucleic acid sequence encoding a polypeptide according to claim 2 is operably linked to a regulatory sequence.

23. A nucleic acid according to claim 22, wherein the regulatory sequence consists of an inducible promoter.

24. The nucleic acid sequence according to claim 23, wherein the regulatory sequence consists of a promoter inducible by an inducer such as Ponasterone.

25. A recombinant vector comprising a nucleic acid sequence as defined in claims 14 to 24.

26. A recombinant host cell comprising a nucleic acid as defined in claims 14 to 24.

27. A recombinant host cell comprising a recombinant vector according to claim 25.

28 A recombinant host cell according to claim 26 or 27 which is eukaryotic.

29. The recombinant host cell according to anyone of claims 26 to 28 which is selected from the group of recombinant hosts cell consisting of zygotes, embryonic, foetal or adult pluripotent stem cells, recombinant hosts cell derived from mammal blastomeres or blastocysts and ES cells.

30. A knock out animal generated by the use of a vector comprising a polynucleotide construct with a sequence comprising the portion of the full length PDE7 which remains after deletion of a nucleic acid according to anyone of claims 14 to 24, said construct replacing thus a portion of the naturally occuring PDE7 sequence within the genome of this animal.

31. A knock out animal according to claim 30 of mammalian origin.

32. A knock out animal according to claim 31 of murine or rat origin.

33. A method for producing a polypeptide as defined in claims 1 to 13, wherein said method comprises the steps of:
a) culturing, in an appropriate culture medium, a recombinant host cell as defined in claims 26 to 29;
b) harvesting the medium thus conditioned or lyse the recombinant host cell;
c) separating or purifying, from the said culture medium, or from the resultant cell lysate, the thus produced polypeptide.

34. A method for the in vitro screening of a compound that inhibits PDE7 phosphodiesterase activity, wherein said method comprises the steps of:
a) providing a desired amount of a polypeptide as defined in claims 1 to 13;
b) adding the desired amount of a polypeptide provided in step a) to a buffer solution containing a desired amount of a candidate compound to be assayed;
c) measuring the phosphodiesterase enzyme activity; and
d) comparing the measured enzyme activity obtained at step c) with the enzyme activity obtained in the absence of the candidate compound.

35. A method according to claim 34 wherein the polypeptide provided at step a) consists of the amino acid sequence beginning at the amino acid residue in position 45 and ending at the amino acid residue in position 427 of SEQ ID No 1, or a homologous polypeptide thereof.

36. A method according to claim 34 or 35 wherein the polypeptide originates from the cell lysate of a host cell transfected with a nucleic acid as defined in claims 14 to 24 or with a recombinant vector according to claim 25.

37. A method according to claim 34 or 36, wherein the phosphodiesterase catalytic activity is measured as the level of hydrolysis of cAMP.

38. A method for the in vitro screening of a compound that inhibits PDE7 phosphodiesterase activity, wherein said method comprises the steps of:
a) Cultivating a recombinant host cell as defined in claims 26 to 29 in an appropriate culture medium;
b) adding a desired concentration of the candidate compound to be assayed in said culture medium;
c) measuring the intracellular phosphodiesterase enzyme activity; and
d) comparing the enzyme activity obtained at step c) with the enzyme activity obtained when step b) is omitted.

39. The method according to claim 38, wherein the recombinant host cell consists of a CHO cell line expressing the dimeric ecdysone receptor.

40. The method according to claim 38, wherein the recombinant host cell has been transfected with a recombinant vector comprising a nucleic acid encoding a polypeptide as defined in claims 1 to 13 which is operably linked to an inducible regulatory sequence.

41. The method according to claim 40, wherein the regulatory sequence is inducible by an inducer.

42. The method according to claim 41, wherein the inducer is Ponasterone.

43. The method according to anyone of claims 38 to 42, wherein before step b), the recombinant hosts cell are cultivated in the presence of the inducer which activates the inducible regulatory sequence during step a).

44. The method according to anyone of claims 38 to 43, wherein an adenylate cyclase activator is added at the end of step b), before the addition of a stop solution.

45. The method according to anyone of claims 38 to 44, wherein the phosphodiesterase enzyme activity is measured after cell lysis through the measure of the amount of intracellular cAMP produced during step b).

46. A method for the in vitro screening of a compound that inhibits PDE7 activity, wherein said method comprises the steps of:
a) providing a recombinant host cell coexpressing a polypeptide according to anyone of claims 1 to 13 and a reporter gene, in an appropriate culture medium;
b) adding a desired concentration of the candidate compound to be assayed in said culture medium;
c) measuring the reporter gene expression; and
d) comparing the enzyme activity obtained at step c) with the enzyme activity obtained when step b) is omitted.

47. The method according to claim 46, wherein the recombinant host cell consists of a CHO cell line expressing the dimeric ecdysone receptor.

48. The method according to claim 46 or 47, wherein the recombinant host cell has been transfected with a recombinant vector comprising a nucleic acid encoding a polypeptide as defined in claims 1 to 13 which is operably linked to an inducible regulatory sequence.

49. The method according to claim 48, wherein the regulatory sequence is inducible by an inducer such as Ponasterone.

50. The method according to anyone of claims 46 to 49, wherein before step b), the recombinant hosts cell are cultivated in the presence of the inducer which activates the inducible regulatory sequence during step a).

51. The method according to anyone of claims 46 to 50, wherein an adenylate cyclase activator is added at the end of step b), before the addition of a stop solution.

52. the method of claims 46 to 51 wherein the reporter gene is a beta lactamase gene.

53. A kit for the in vitro screening of a compound that inhibits PDE7 phosphodiesterase catalytic activity, wherein said kit comprises:
a) a polypeptide as defined in claims 1 to 13;
b) optionally, the reagents necessary to perform the phosphodiesterase catalytic activity measures.

54. A kit for the in vitro screening of a compound that inhibits PDE7 phosphodiesterase catalytic activity, wherein said kit comprises:
a) a recombinant host cell as defined in claims 26 to 29;
b) optionally, the reagents necessary to perform the phosphodiesterase catalytic activity measurement.

55. A kit for the in vitro screening of a compound that inhibits PDE7 phosphodiesterase activity, wherein said kit comprises:
a) a recombinant host cell of the invention coexpressing a polypeptide as defined in claims 1 to 13 and a reporter gene; and
b) optionally, the reagents necessary to perform the reporter gene expression measurement.

56. A method for selecting in vitro a compound that inhibits PDE7 phosphodiesterase activity, wherein said method comprises the steps of:
a) performing the method as defined in claims 34 to 37 with a candidate compound; and in case that said candidate compound is found to inhibit the phosphodiesterase activity, then
b) performing the method as defined in claims 38 to 52 with the inhibitor compound selected at step a)

57. A method for selecting a compound that selectively inhibits PDE7 phosphodiesterase activity, wherein said method comprises the steps of:
a) selecting a compound which inhibits PDE7 phosphodiesterase activity by carrying out a method as defined in claims 34 to 37, 38 to 52 and 56; and
b) assaying the selected inhibitor compound for its inability to inhibit the phosphodiesterase activity of at least one PDE enzyme other than PDE7.

58. The method according to claim 57, wherein the selected inhibitor is assayed for its inability to inhibit the phosphodiesterase activity of PDE3 and PDE4 enzymes.

59. The method according to claim 57, wherein the selected inhibitor is assayed for its inability to inhibit the phosphodiesterase activity of PDE1, PDE2, PDE3, PDE4, PDE5 and PDE6 enzymes.

60. A phosphodiesterase inhibitor compound selected according to a method as defined in claims 34 to 37, 38 to 52, 56 and 57 to 59.

61. The use of a phosphodiesterase inhibitor compound selected according to a method as defined in claims 34 to 37, 38 to 52, 56 and 57 to 59. for manufacturing a pharmaceutical composition.

62. The use of a compound obtainable identifiable, selectable, or characterizable by the method according to anyone of claims 34 to 52 and 56 to 59 for the manufacture of a pharmaceutical composition to be used in the treatment, diagnosis or surgery of the human or animal.

64. The use of a compound obtained, identified, selected or characterized by the method according to anyone of claims 34 to 52 and 56 to 59 for the manufacture of a pharmaceutical composition to be used in the treatment, diagnosis or surgery of the human or animal.

65. The use of a compound obtained, identified, selected or characterized by the method according to anyone of claims 34 to 52 and 56 to 59 for the manufacture of a pharmaceutical composition for the treatment or prevention of various pathological conditions such as diseases affecting the immune system, including AIDS, rejection of transplant, auto-immune disorders such as T-cells related diseases for example rheumatoid arthritis; inflammatory diseases such as respiratory inflammation diseases including chronic obstructive pulmonary disease (COPD), asthma; gastrointestinal inflammation diseases such as Crohn's disease, colitis, pancreatitis as well as different types of cancers including leukaemia.

66. The use of a compound obtainable, identifiable, selectable or characterizable by the method according to anyone of claims 34 to 52 and 56 to 59 for the manufacture of a pharmaceutical composition for the treatment or prevention of various pathological conditions such as diseases affecting the immune system, including AIDS, rejection of transplant, auto-immune disorders such as T-cells related diseases for example rheumatoid arthritis; inflammatory diseases such as respiratory inflammation diseases including chronic obstructive pulmonary disease (COPD), asthma; gastrointestinal inflammation diseases such as Crohn's disease, colitis, pancreatitis as well as different types of cancers including leukaemia

67. The use of a compound obtained, identified, selected or characterized by the method according to anyone of claims 34 to 52 and 56 to 59 in the synthesis of a compound for the treatment or prevention of various pathological conditions such as diseases affecting the immune system, including AIDS, rejection of transplant, auto-immune disorders such as T-cells related diseases for example rheumatoid arthritis; inflammatory diseases such as respiratory inflammation diseases including chronic obstructive pulmonary disease (COPD), asthma; gastrointestinal inflammation diseases such as Crohn's disease, colitis, pancreatitis as well as different types of cancers including leukaemia


SEQUENCE LISTING (35 sequences)

SEQ ID NO: 1 - protein, 426 aa, Homo sapiens
DQTALYIRMLGDVRVRSRAGFESERRGSHPYIDFRIFHSQSEIEVSVSARNIRRLLSFQRYLRSSRFFRGTAVSNSLNILDDDYNGQAKCMLEKVGNWNFDIFLFDRLTNGNSLVSLTFHLFSLHGLIEYFHLDMMKLRRFLVMIQEDYHSQNPYHNAVHAADVTQAMHCYLKEPKLANSVTPWDILLSLIAAATHDLDHPGVNQPFLIKTNHYLATLYKNTSVLENHHWRSAVGLLRESGLFSHLPLESRQQMETQIGALILATDISRQNEYLSLFRSHLDRGDLCLEDTRHRHLVLQMALKCADICNPCRTWELSKQWSEKVTEEFFHQGDIEKKYHLGVSPLCDRHTESIANIQIGFMTYLVEPLFTEWARFSNTRLSQTMLGHVGLNKASWKGLQREQSSSEDTDAAFELNSQLLPQENRLS

SEQ ID NO: 2 - protein, 426 aa, Mus sp.
DQTALYIRMLGDVRVRSRAGFETERRGSHPYIDFRIFHSQSDIEASVSARNIRRLLSFQRYLRSSRVFRGATVCSSLDILDEDYNGQAKCMLEKVGNWNFDIFLFDRLTNGNSLVSLTFHLFSLHGLIEYFHLDMVKLRRFLVMIQEDYHSQNPYHNAVHAADVTQAMHCYLKEPKLASSVTPWDILLSLIAAATHDLDHPGVNQPFLIKTNHYLATLYKNSSVLENHHWRSAVGLLRESGLFSHLPLESRQEMEAQIGALILATDISRQNEYLSLFRSHLDKGDLHLDDGRHRHLVLQMALKCADICNPCRNWELSKQWSEKVTEEFFHQGDIEKKYHLGVSPLCDRQTESIANIQIGFMTYLVEPLFTEWARFSATRLSQTMLGHVGLNKASWKGLQRQQPSSEDANAAFELNSQLLTQENRLS

SEQ ID NO: 3 - protein, 426 aa, Rattus sp.
DQTALYIRMLGDVRVRSRAGFETERRGSHPYIDFRIFHAQSEIEASVSARNIRRLLSFQRYLRSSRFFRGATVCRSLNILDEDYNGQAKCMLEKVGNWNFDIFLFDRLTNGNSLVSLTFHLFSLHGLIEYFHLDMVKLRRFLVMIQEDYHSQNPYHNAVHAADVTQAMHCYLKEPKLANSVTPWDILLSLIAAATHDLDHPGVNQPFLIKTNHYLATLYKNTSVLENHHWRSAVGLLRESGLFSHLPLESRHEMEAQIGALILATDISRQNEYLSLFRSHLDKGDLHLDDGRHRHLVLQMALKCADICNPCRNWELSKQWSEKVTEEFFHQGDIEKKYHLGVSPLCDRQTESIANIQIGFMTYLQEPLFTEWARFSDTRLSQTMLGHVGLNKASWKGLQRQQPSSEDASAAFELNSQLLTQENRLS

SEQ ID NO: 4 - DNA, 1281 bp, Homo sapiens
gatcagactgcattatacattcgtatgctaggagatgtacgtgtaaggagccgagcaggatttgaatcagaaagaagaggttctcacccatatattgattttcgtattttccactctcaatctgaaattgaagtgtctgtctctgcaaggaatatcagaaggctactaagtttccagcgatatcttagatcttcacgcttttttcgtggtactgcggtttcaaattccctaaacattttagatgatgattataatggacaagccaagtgtatgctggaaaaagttggaaattggaattttgatatctttctatttgatagactaacaaatggaaatagtctagtaagcttaacctttcatttatttagtcttcatggattaattgagtacttccatttagatatgatgaaacttcgtagatttttagttatgattcaagaagattaccacagtcaaaatccttaccataacgcagtccacgctgcggatgttactcaggccatgcactgttacttaaaggaacctaagcttgccaattctgtaactccttgggatatcttgctgagcttaattgcagctgccactcatgatctggatcatccaggtgttaatcaacctttccttattaaaactaaccattacttggcaactttatacaagaatacctcagtactggaaaatcaccactggagatctgcagtgggcttattgagagaatcaggcttattctcacatctgccattagaaagcaggcaacaaatggagacacagataggtgctctgatactagccacagacatcagtcgccagaatgagtatctgtctttgtttaggtcccatttggatagaggtgatttatgcctagaagacaccagacacagacatttggttttacagatggctttgaaatgtgctgatatttgtaacccatgtcggacgtgggaattaagcaagcagtggagtgaaaaagtaacggaggaattcttccatcaaggagatatagaaaaaaaatatcatttgggtgtgagtccactttgcgatcgtcacactgaatctattgccaacatccagattggttttatgacttacctagtggagcctttatttacagaatgggccaggttttccaatacaaggctatcccagacaatgcttggacacgtggggctgaataaagccagctggaagggactgcagagagaacagtcgagcagtgaggacactgatgctgcatttgagttgaactcacagttattacctcaggaaaatcggttatcataa

SEQ ID NO: 5 - DNA, 1281 bp, Mus sp.; identical to SEQ ID NO: 4

SEQ ID NO: 6 - DNA, 1281 bp, Rattus sp.
gatcagactgcgttatatattcgtatgctaggagatgtccgagtgaggagccgagcaggatttgagacagaaagaagaggttcccacccgtacattgacttccgtatttttcacgctcaatctgaaattgaagcatccgtctccgccagaaacatcagaaggctgctcagtttccagcggtatctcagatcctcacgattttttcggggtgccacagtttgtagatctttaaatattttagatgaggattacaatggacaagccaagtgtatgctggaaaaagttggaaattggaattttgatatctttctgtttgatagactaacaaatggaaatagtctagtaagcttaacctttcatttatttagtcttcatggattgattgagtacttccatttagatatggtgaaactccgtcgatttttagttatgattcaagaagattaccacagtcaaaatccttaccacaatgcagtccatgctgcagatgttacccaggccatgcactgttacttaaaggaacctaagcttgccaattctgtaactccttgggatatcttgctgagcttaattgcagctgccactcatgatctggatcacccaggtgttaatcagccatttctcattaaaaccaaccattacttggcaactttatacaagaatacctcagtcctggagaatcaccactggagatctgccgtgggcttgttgagagaatctggtctgttctcacacttgccattggaaagcaggcatgagatggaggctcagataggtgctttgatactagccacggacatcagtcgccagaatgagtacctgtcattgtttagatctcacttggataaaggtgacttacaccttgacgatggcagacataggcatttggttctacagatggccttgaaatgtgctgatatttgtaacccatgtcggaactgggaattaagcaagcagtggagtgaaaaagtaacggaggaattcttccaccaaggcgatatagaaaaaaagtaccatttgggtgtgagtccactttgtgatcgtcagactgagtctattgccaacatccagattggttttatgacttaccttcaggagcctttatttacagagtgggccaggttttcagacacgaggctgtcacagacgatgctcggacacgtggggctgaataaagccagctggaagggactacaaagacaacagcctagcagcgaggatgccagtgctgcatttgagttgaactcacagctattaactcaggaaaatcggttatcataa

SEQ ID NO: 7 - DNA, 20 bp, Artificial Sequence
  Description of Artificial Sequence Primer
aaatggaaatagtctagtaa

SEQ ID NO: 8 - DNA, 25 bp, Artificial Sequence
  Description of Artificial SequencePrimer
aattcttgtataaagttgctagata

SEQ ID NO: 9 - DNA, 24 bp, Artificial Sequence
  Description of Artificial Sequence Probe
acatgggttacaaatatcagcaca

SEQ ID NO: 10 - DNA, 31 bp, Artificial Sequence
  Description of Artificial Sequence Probe
ctttatttacagagtgggccaggttttcagc

SEQ ID NO: 11 - DNA, 3165 bp, Mus sp.
ctctcttcgccctgggagggctgttattcaccggactccttcaggagagaggatgtggtggacctctggttgatccaatgggaatcactttgatctggtgtctggctttggttctgatcaagtggatcacctctaagagacgtggagctatttcctatgacagttctgatcagactgcgttatatattcgtatgctaggagatgtccgcgtgaggagccgagcaggatttgaaacagaaagaagaggttcccatccgtacattgacttccgtatttttcactctcaatctgacattgaagcatcagtctccgccaggaacatcagaaggttactaagtttccagagatatctcagatcctcacgagtttttcggggtgccacagtttgtagttctctagacattttagatgaggattacaatggacaagccaagtgtatgctggaaaaagttggaaattggaattttgatatctttctgtttgatagactaacaaatggaaatagtctagtaagcctaacctttcatttatttagtcttcatggattgattgagtacttccatttagatatggtgaaactccgtcggtttttagttatgattcaagaagattaccacagtcaaaatccttaccacaatgcagtccatgctgcagacgttactcaggccatgcactgttacttaaaggaacctaagcttgccagttctgtcactccttgggatatcttgctgagtttaattgcagccgccactcacgatctggatcacccaggtgttaatcagccgtttcttattaaaaccaaccattatctagcaactttatacaagaattcctcagtcctggagaatcaccactggagatctgcagtgggcttgttaagagaatctggtttgttctcacacttgccattggaaagcaggcaagagatggaggctcagataggtgctttgatattagccacggatatcagtcgccagaacgagtacctgtcattgtttagatctcacttggataaaggtgacttacaccttgacgatggcagacataggcatttggttctacagatggccttgaaatgtgctgatatttgtaacccatgtcggaactgggaattaagcaagcagtggagtgaaaaagtaacggaggaattcttccaccaaggagatatagaaaaaaagtaccatttgggtgtgagtccactttgtgatcgtcagactgagtctattgccaacatccagattggttttatgacttacctagtggagcctttatttacagagtgggccaggttttcagccacacggctgtcacagacgatgcttggacatgtggggctgaataaagccagctggaagggactgcagagacaacagcccagcagcgaggatgccaatgctgcatttgagttgaactcacagttactaactcaggaaaatcggttatcataaccctgaaccagtgggacaactccctcctgcaacattgtggaaatgtgaaatggtcttgagtggaagaactggttctgactgctgaggtttccaaggggatgatgccagcattatctatcaagatgtcttctgttagatcatacgagcgcacgttttaatggtagacatgaatatacagcaatatgcatcttgcttttgtatgaaaccttaagtctgtaaagctcagcaggagaaccacaaagggttcttaacgaaggaagatgatgttttaggtgtgccatgtgctttcagagtgtgtgtccttcaagccctgaaacttgaactcaacctcagcagcaatcttgatgcaaccagtccagtccaacagcgcatccgtcccttccactacattctctctgagaaaacagaaatgtgaagtgcccagcctctgccctctgcaagaatcaatcaaatgtttacaaatcaaatctgaactattggttctaaattgcctcggagcatcgttggaaggaaccactgaccgttctcagctgagctggagactgcaagctcttcgcagcatttgccttttcttcttccgatgtcaatgtcatcatcgcctcccacctgctgctctgctctgtgcctggagactgagcatttatttaccacagacgccacacgaagcttttcactccagcgtggttggcaatgcagattccttttttaactgttagtttttaacatggggtgggagggaaggaacaccactgtcctagctgtgttctgactctgcagtgaagacactgcatgttgttttcactactatacacttgacctacacatgcaaggaaaaggtggcttgttttccattccataaggagctcaggggatttgccaatctgaaataaagtgggctagggaagtgtgtccttcaaatcaagggtactaaagtccctttcgctgcagtgtgtgagaggtatgttgtgtatggctgtatggatgtgtgtttgcgtgcatgtttgtgcatgtatggctgtgcatgttccgtccatccatgtgggaaaggcttagaagtgtaagttcttatttattgttctagaatgtgacatagtgagcagccacacttgggggaggggaaggtaggtgaagctgtaagattgctccagatgccttaaactatgcacaaagctaagtgaccaaacttctcattttgatttgaaaagtgcattgttttcttgtcccccctcccccactttgatgaaacattaccctttggcgaccttttgatgtgcagtagatcgttttctaggataaaatttaaggactaattttagacttaaaacattccacttttgtaagttgttttcagtggtcttatgtagagcaagcacaactgtaacctagtttaagagaaacggtgctttcttttagttaccttctatttcccttgttactgtttaagactgttaattatgtttacagtttgttgcaacagccatcttgggagagaacttgagtgtaaagccatgtgtgaaggctctgggcacactcgtttaaacacgtgcctgccgcttcaccttctgattgacaaataaaaccggtctttgctcattggttaacctttgcactctaccttaccactcagtcctgttggaagtgacatgctggattcttaggtgatcagttagctgctgcctctcccgttcagtcctgggtattttgaagtctaagttgt

SEQ ID NO: 12 - DNA, 27 bp, Artificial Sequence
  Description of Artificial SequencePrimer
cggggatccatggaagtgtgttaccag

SEQ ID NO: 13 - DNA, 37 bp, Artificial Sequence
  Description of Artificial SequencePrimer
cgcgtctagattatgataaccgattttcctgaggtaa

SEQ ID NO: 14 - DNA, 30 bp, Artificial Sequence
  Description of Artificial SequencePrimer
cgggatccgccaccatggaagtgtgttacc

SEQ ID NO: 15 - DNA, 36 bp, Artificial Sequence
  Description of Artificial SequencePrimer
ccgggggtaccggcggccgcggcagggcgggcgccg

SEQ ID NO: 16 - DNA, 32 bp, Artificial Sequence
  Description of Artificial SequencePrimer
gggggatcctgaatacgcccgccctgcctccg

SEQ ID NO: 17 - DNA, 39 bp, Artificial Sequence
  Description of Artificial SequencePrimer
gggtctagaccccagaaccagtgggacaaactgcctcct

SEQ ID NO: 18 - DNA, 34 bp, Artificial Sequence
  Description of Artificial SequencePrimer
ggcgggccctggagaaacataacatgcacgtcac

SEQ ID NO: 19 - DNA, 36 bp, Artificial Sequence
  Description of Artificial SequencePrimer
ccgggatccgacaggccggtcccccagcacgtcctc

SEQ ID NO: 20 - DNA, 34 bp, Artificial Sequence
  Description of Artificial SequencePrimer
cgcgtctagattatgataaccgattttcctgagg

SEQ ID NO: 21 - DNA, 33 bp, Artificial Sequence
  Description of Artificial SequencePrimer
ccgggatccccccggcagctctctcagaggcgt

SEQ ID NO: 22 - DNA, 31 bp, Artificial Sequence
  Description of Artificial SequencePrimer
ccgggatccttatacattcgtatgctaggag

SEQ ID NO: 23 - DNA, 31 bp, Artificial Sequence
  Description of Artificial SequencePrimer
ccgggatccagaagaggttctcacccatata

SEQ ID NO: 24 - DNA, 37 bp, Artificial Sequence
  Description of Artificial SequencePrimer
ccggggatccgtgtctgtctctgcaaggaatatcaga

SEQ ID NO: 25 - DNA, 35 bp, Artificial Sequence
  Description of Artificial SequencePrimer
ccggggatccatgtcacgcttttttcgtggtactg

SEQ ID NO: 26 - DNA, 33 bp, Artificial Sequence
  Description of Artificial SequencePrimer
ccggggatccaatggacaagccaagtgtatgct

SEQ ID NO: 27 - DNA, 34 bp, Artificial Sequence
  Description of Artificial SequencePrimer
ccggggatccatggtaagcttaacctttcattta

SEQ ID NO: 28 - DNA, 30 bp, Artificial Sequence
  Description of Artificial SequencePrimer
cgcgtctagattagctggctttattcagcc

SEQ ID NO: 29 - DNA, 35 bp, Artificial Sequence
  Description of Artificial SequencePrimer
cgcgtctagattacctggcccattctgtaaataaa

SEQ ID NO: 30 - DNA, 36 bp, Artificial Sequence
  Description of Artificial SequencePrimer
cgcgtctagattataggtaagtcataaaaccaatct

SEQ ID NO: 31 - DNA, 19 bp, Artificial Sequence
  Description of Artificial Sequence oligonucleotide
gggtttactgtgctctcca

SEQ ID NO: 32 - DNA, 19 bp, Artificial Sequence
  Description of Artificial Sequence oligonucleotide
tgccaccacactctgctct

SEQ ID NO: 33 - DNA, 22 bp, Artificial Sequence
  Description of Artificial Sequence oligonucleotide
aaaaactaactaactaactaaa

SEQ ID NO: 34 - DNA, 22 bp, Artificial Sequence
  Description of Artificial Sequence oligonucleotide
cttctatacattggcttgattg

SEQ ID NO: 35 - DNA, 2939 bp, Mus sp.
  unsure (1428): Unknown
gaattcctccgttactttttcactccactgcttgcttaattcccagttccgacatgggttacaaatatcagcacatttcaaggccatctagcaaacaaaatattaaaagggtgttaaggaagaaaagatctcattgtaccaagtttaaatcgactccagcaatggggaagagcactgaagagagagaggagcccctgacatgttgtgttcagtgtgcatgggatccagaaccaagtaaaattaaaagccagcaaagaagaggaaaacactgtgcaagaaaaatcactgtgacattaacttcatgctgctactgatctaccaagtatcggtgcagaaggctcacagggagctaactctgtagaattacccttgacaatccaaaggagacacaatgactgaccagtcagaaaatacaaacaaatccagagacaccatatactccttgcccctcttttcatgaggaaactcatcagatagtcactgttgactggacggcattctcagaamcctgagtaggaaaagtcccaaagccacatgcctctcctgtatgttctggaagggaacgtgctgtctgtggaacctccttcctcccaacttaccctgaaaaaaagatctgctatatttcatccttgttctacctgtcacatgatttaccctggaaacaatctttgcaagttgcagaactttacctgtagaaccaaatgcctatgtctgccatcgtcaaggtgtaagtcacctttatccaagtgagatctaaacaatgacaggtactcgttctggcgactgatatccgtggctaatatcaaagcacctatctgagcctccatctcttgcctggaattacagaaataatgagacatttagtactttcaatttattttctctcttaagtttaagtattcagaaagctttacaaaaccctaagatactctgaaagttgctttgaacttgatatataagaaagtattatgaagagtgcatttaaaaatattaaaataactaaagtaaaactaagagttttcagggaaatactacaatgcaaagatgcccaaggaagcgtctttatgcgtcagcccaaagtcaaattatttgcttaaaaggagggaacaaccmacctaataccattatgctgtttgagaaaattcttaagaattttctgagatattcttgtatgtataacaaacaacctttgtcytattttttggtgcctaggacctaaaaaaagattcaggcagagaaaatattaattgtctagtgagacatctgagaaagatgcttaatgcttaatgaaataagcatcttaataaaattctactcagcagtttgatcaaggggccagagatgggttcagcaatttatggtataggctctttttctagaggttggttcccaactccctcatggcagcctacccataaggtaattccnttttcagagagttcctcactctctcctggctctgtgctacccaccatggatgtggtgcatggatatacatgtaagcnaaatacacatgcacctaaaataatcaagagtcagttatctgtgtaagaatattcaaatttatctggaggaaaaatgtcacatgttaatacagacagcacatatactgcatctttttaaaaacggcctatatgagatttaaaaaaaaaaaaccctcacagagtcacaataagattatcttggtattaatggtaacaagaagaccnttgagatggctcacaggagtccttgcctcntgagcctgatggnctagaggtcatccttgggaccccagaggaagcagaccaaaggwctgctgaaggcaatcctcagacctgttgtgnccacagcacacctgttgctgagtncacacacacacacacacattttagtcacattcggaaaaanccaanacaggttcaaacaaacncaanactgaatatttacacacaaagtttaaaatccangccggcaaggtttnaaatgtttttgccaacaccatcatattggacttatttaactttagtgtttcttctnatgccaccaaaatgnccttagtccttagtattaaattattttttaagctattttctgttgttttattcattatccttcacgacagctccaaagcgtacatnagggcacaggnccttattatccttgttttactggctttgcatatgttgtggtctgaatatatcttgttatgcactgtcnctttctgtgtaatctttcaggtatgcacctgaaccctttattagaaaaggtacgtacacccantgagccattatgtgtgaagtgacttttcacagtactcaaaaaggggtatctttcataaaaataaatagccaatgtaaaagcgacaactacgtagcaggcttaagaatttgggtataacttcctttggatttccagtgtatctgtgtttggctcacatttatgtatgtgtgccatgtgtagatatctgtgagctatcatgtggggagctgggaactgaacccagatcttctcctagaacagcaaaaattctaaactgntgagccntttntccagcaccacaattttgataattttgttgttgttgttgtttgtttgttttgcttctngagacagggtttttctgtatagccctggctgtcctggaactcantctgtagaccaggctggcctngantcagaaatctgcctgcttctgcctcccaagagctgggattaaaggcgtgccacgcccagctaatcttgatttttaaaaagatacgtttagtttaatgaaacgggaagctatggagcagctgaataacatatttttatcttaattagtaactttatttccataggcaatgcatttgttttaagccaccttacctgctttccaatggcaagtgtgagaacaaaccagattctcttaacaagcccactgcagatctccagtggtgttctccaggactgaggaattc